SEQUENCE LISTING (22 sequences)

SEQ ID NO: 1 - protein, 278 aa, Bos taurus
  MOTIF 152..154: RGD
MRIAVICFCLLGIASALPVKPTSSGSSEEKQLNNKYPDAVATWLKPDPSQKQTFLAPQNSVSSEETDDNKQNTLPSKSNESPEQTDDLDDDDDNSQDVNSNDSDDAETTDDPDHSDESHHSDESDEVDFPTDIPTIAVFTPFIPTESANDGRGDSVAYGLKSRSKKFRRSNVQSPDATEEDFTSHIESEEMHDAPKKTSQLTDHSKETNSSELSKELTPKAKDKNKHSNLIESQENSKLSQEFHSLEDKLDLDHKSEEDKHLKIRISHELDSASSEVN

SEQ ID NO: 2 - protein, 314 aa, Homo sapiens
  Human OPN
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 3 - protein, 7 aa, Bos taurus
SVAYGLK

SEQ ID NO: 4 - protein, 7 aa, Homo sapiens
SVVYGLR

SEQ ID NO: 5 - protein, 7 aa, Mus musculus
SLAYGLR

SEQ ID NO: 6 - protein, 7 aa, Oryctolagus
SVAYRLR

SEQ ID NO: 7 - protein, 5 aa, Bos taurus
GDSVA

SEQ ID NO: 8 - protein, 5 aa, Homo sapiens
GDSVV

SEQ ID NO: 9 - protein, 5 aa, Mus musculus
GDSLA

SEQ ID NO: 10 - protein, 5 aa, artificial sequences
  artificial penta peptide
GDSLV

SEQ ID NO: 11 - protein, 16 aa, Bos taurus
  Fragment from bovine OPN
IPTESANDGRGDSVAY

SEQ ID NO: 12 - protein, 17 aa, Bos taurus
IPTESANDGRGDSVAYG

SEQ ID NO: 13 - protein, 18 aa, Bos taurus
IPTESANDGRGDSVAYGL

SEQ ID NO: 14 - protein, 19 aa, Bos taurus
IPTESANDGRGDSVAYGLK

SEQ ID NO: 15 - protein, 21 aa, Bos taurus
IPTESANDGRGDSVAYGLKSR

SEQ ID NO: 16 - protein, 9 aa, Bos taurus
GDSVAYGLK

SEQ ID NO: 17 - protein, 11 aa, Bos taurus
GDSVAYGLKSR

SEQ ID NO: 18 - protein, 9 aa, Bos taurus
GDSVAYGLK

SEQ ID NO: 19 - protein, 25 aa, Bos taurus
IPTESANDGRGDSVAYGLKSRSKKF

SEQ ID NO: 20 - protein, 6 aa, Bos taurus
  Fragment from bovine OPN
GDSVAY

SEQ ID NO: 21 - protein, 5 aa, Bos taurus
VAYGL

SEQ ID NO: 22 - protein, 8 aa, Bos taurus
  VARIANT 1: Ala
GDSVAYGL

(19) United States
(12) Patent Application Publication
(10) Pub. No.: US 2015/0190460 A1
(43) Pub. Date: Jul. 9, 2015

(54) OSTEOPONTIN PEPTIDE FRAGMENTS FOR USE IN SUPPRESSION OR PREVENTION OF TUMOR GROWTH

(71) Applicants: FORSYTH DENTAL INFIRMARY FOR CHILDREN (D/B/A THE FORSYTH INSTITUTE), CAMBRIDGE, MA (US); ARLA FOODS AMBA, VIBY J (DK)

(72) Inventors: Susan R. Rittling, Brookline, MA (US); Peter Langborg Wejse, Aarhus N (DK); Anja Serena, Bjerringbro (DK)

(21) Appl. No.: 14/415,431

(22) PCT Filed: Jul. 19, 2013
(86) PCT No.: PCT/EP2013/065320

§ 371 Date: Jan. 16, 2015

Related U.S. Application Data
(60) Provisional application No. 61/674,016, filed on Jul. 20, 2012.

(30) Foreign Application Priority Data
Jul. 20, 2012 (EP) 12177351.9
Jun. 12, 2013 (EP) 13171711.8

(51) Int. Cl. A61K 38/08; A23L 1/305; C07K 7/06

(52) U.S. Cl. CPC A61K 38/08; C07K 7/06; A23L 1/3053; A23V 2002/00

(57) ABSTRACT

The present invention relates to isolated, pharmaceutically active, osteopontin-related molecules, pharmaceutical compositions and nutritional supplements comprising such molecules, and use of such compositions and supplements for treating or preventing tumor-generating cancer.

### FIELD OF THE INVENTION

The present invention pertains to isolated, pharmaceutically active, molecules related to osteopontin-derived peptide fragments, pharmaceutical compositions and nutritional supplements comprising such active molecules, and use of such compositions and supplements for treating or preventing tumor-generating cancer.

### BACKGROUND OF THE INVENTION

Osteopontin (OPN) is a secreted, adhesive glycophosphoprotein originally isolated from the collagenous extracellular matrix of mineralized bone (Franzen 1985). Osteopontin is expressed by a number of different cell types, including osteoblasts, arterial smooth muscle cells, leukocytes, several types of epithelial cells, and transformed cells of different lineages (Denhardt 1995). Accordingly, OPN has been detected in many tissues, including kidney, placenta, secretory epithelia and ganglia of the inner ear, and smooth muscle of the vascular system (Butler 1996). Osteopontin is also present in many body fluids, for example plasma, urine, bile and milk, and it displays elevated expression in many transformed cells (Senger 1988). This protein is highly acidic with approximately 25% of the amino acid being aspartate/aspartic acid and glutamate/glutamic acid, and OPN has a significant number of phosphorylated amino acids (Sorensen 1994).

### SUMMARY OF THE INVENTION

The present inventors have made the surprising discovery that orally administered osteopontin suppresses, and possibly even prevents, the growth of cancer tumors. This effect was completely unexpected. First of all, administration of OPN has never been documented to have beneficial effects relating to the treatment of cancer, and secondly, it is very surprising that an orally administered protein has medical effects outside the gastrointestinal system.

The inventors have subsequently performed further pre-clinical trials (see examples 6 and 7) and have demonstrated that small peptide fragments related to OPN also suppress the growth of cancer tumors.

Thus, an aspect of the invention pertains to an isolated, pharmaceutically active, molecule comprising an amino acid sequence of at least three amino acid residues from:
position 153 to position 160 of SEQ ID NO. 1 or from an amino acid sequence containing one or more conservative amino acid substitutions relative to position 153 to position 160 of SEQ ID NO. 1, or
position 160 to position 167 of SEQ ID NO. 2 or from an amino acid sequence containing one or more conservative amino acid substitutions relative to position 160 to position 167 of SEQ ID NO. 2.

Another aspect of the invention pertains to an isolated, pharmaceutically active, osteopontin-derived (OPN-derived) peptide fragment comprising a sequence of at least two amino acid residues from position 140 to position 170 of SEQ ID NO. 1 or from position 147 to position 177 of SEQ ID NO. 2.

Yet an aspect of the invention pertains to an isolated, pharmaceutically active, OPN-derived peptide fragment as defined herein or the isolated, pharmaceutically active, molecule as defined herein, for use as a medicament. For example, the invention may pertain to an active agent comprising one or more isolated, pharmaceutically active, molecules as defined herein, for use as a medicament.

Another aspect of the invention pertains to an active agent comprising, or even consisting of, one or more isolated, pharmaceutically active, OPN-derived peptide fragment(s) as defined herein or the isolated, pharmaceutically active, molecule as defined herein, for use in the treatment or prevention of cancer involving at least one cancer tumor.

In the context of the present invention, the term “cancer involving a cancer tumor” pertains to cancer diseases during which at least one cancer tumor is formed in or on the patient. The at least one cancer tumor may be the primary cancer tumor of the cancer or a subsequent metastasis.

A further aspect of the invention pertains to a method of treating or preventing cancer, the method comprising: administering to a subject having cancer, or to a subject being at risk of getting cancer, an amount of an active agent comprising, or even consisting of, the one or more isolated, pharmaceutically active, OPN-derived peptide fragment(s) as defined herein or the isolated, pharmaceutically active, molecule as defined herein, which amount is effective to treat or prevent said cancer, and wherein said cancer involves at least one cancer tumor.

Yet another aspect of the invention pertains to a pharmaceutical composition comprising:
an active agent comprising, or even consisting of, the one or more isolated, pharmaceutically active, OPN-derived peptide fragment(s) as defined herein or one or more isolated, pharmaceutically active, molecules as defined herein, and
a pharmaceutically acceptable carrier.

An additional aspect of the invention pertains to a nutritional supplement comprising
a nutritionally effective amount of an active agent comprising, or even consisting of, the one or more isolated, pharmaceutically active, OPN-derived peptide fragment(s) as defined herein or one or more isolated, pharmaceutically active, molecules as defined herein, and
one or more components selected from the group consisting of a carbohydrate source, a lipid source, and a protein source.

Some aspects provided here relate to methods, comprising administering to a subject having a tumor cell mass an amount of the active agent effective to suppress tumor cell growth or replication.

In certain embodiments, the active agent is administered at a concentration of about 0.05 mg/ml to about 1 g/ml. In other embodiments, the active agent is administered in an amount of about 0.05 mg/kg of body weight to about 5 g/kg.

In particular embodiments, the active agent is administered mucosally. In other embodiments, the active agent is administered orally, sublingually, buccally, or nasally.

In some embodiments, the tumor cell may be a subcutaneous tumor cell, and in any one of the foregoing embodiments, the tumor cell may reside in a breast, cervix, ovary, prostate, lung, colon, rectum, pancreas, stomach, kidney, or thyroid.

In certain embodiments, the active agent is isolated from bovine milk.

In particular embodiments, the active agent is recombinant OPN.

In some embodiments, the active agent is purified. In certain embodiments, the purified active agent is at least about 95% pure.

Other aspects provided herein are directed to pharmaceutical compositions that include the active agent, preferably an amount of the active agent effective to suppress tumor cell growth or replication, and a pharmaceutically acceptable carrier.

In certain embodiments, the amount of the active agent in the pharmaceutical composition is about 0.05 mg/ml to about 1 g/ml.

In particular embodiments, the pharmaceutical composition is formulated for mucosal administration. The pharmaceutical composition may be formulated for oral, sublingual, buccal, or nasal administration.

In some embodiments, the pharmaceutically acceptable carrier is selected from the group consisting of ethanol, glycerine, propylene glycol, polyethylene glycol, sugar solution, sorbitol, buffer, saline, and water.

In certain embodiments, the pharmaceutical composition is a solid, a liquid, or an emulsion.

In particular embodiments, the pharmaceutical composition is administered as a tablet or a spray.

In any one of the foregoing embodiments, the pharmaceutical composition may comprise a taste-masking agent.

In any one of the foregoing embodiments, the pharmaceutical composition may comprise one or more anticancer targeted therapeutic or chemotherapeutic agents.

In any one of the foregoing embodiments, the pharmaceutical composition may comprise one or more immunosuppressive or immune-stimulating agents.

Other aspects provided herein are directed to nutritional supplements that include the active agent, preferably an amount of the active agent effective to suppress tumor cell growth or replication.

In some embodiments, the supplement is a liquid or a powder.

In certain embodiments, the supplement comprises one or more fruit(s), one or more vegetable(s), yogurt, milk, ice cream, or a combination thereof.

In any one of the foregoing embodiments, the supplement may be fortified with protein, vitamins, minerals, antioxidants, prebiotics, probiotics, or a combination thereof.

In any one of the foregoing embodiments, the supplement may be lactose-free and/or gluten-free.

In certain embodiments, the supplement is organic.

In some embodiments, the supplement is a smoothie or a juice, while in other embodiments, the supplement is a milk.

These and other aspects of the invention will be described in connection with the drawings and the detailed description below.

### BRIEF DESCRIPTION OF THE DRAWINGS

FIG. 1A shows tumor volume (cm³, +/−SEM) in mice that received oral doses of OPN at various concentrations, starting on day 0.

FIG. 1B shows tumor volume (cm³, +/−SEM) in mice that received oral doses of OPN at various concentrations, starting on day 5.

FIGS. 2A-C show a comparison of tumor sizes in all groups on days 15, 17, and 19; significant differences are indicated.

FIG. 3 shows the mean tumor size of control and OPN-fed mice (0.3 mg/ml in drinking water), combined results from three independent experiments. N=30 (0 mg/ml OPN) or 32 (0.3 mg/ml OPN).

FIG. 4 shows the tumor volume (cm³, +/−SEM) of mice that received administration of peptide and of mice in the control group.

FIG. 5 shows the final tumor weight of mice that received administration of peptide and of mice in the control group.

### DETAILED DESCRIPTION OF THE INVENTION

An aspect of the invention pertains to an isolated, pharmaceutically active, molecule comprising an amino acid sequence of at least three amino acid residues from:
position 153 to position 160 of SEQ ID NO. 1 or from an amino acid sequence containing one or more conservative amino acid substitutions relative to position 153 to position 160 of SEQ ID NO. 1, or
position 160 to position 167 of SEQ ID NO. 2 or from an amino acid sequence containing one or more conservative amino acid substitutions relative to position 160 to position 167 of SEQ ID NO. 2.

In some preferred embodiments of the invention, the amino acid sequence of the active molecule contains at most 15 consecutive amino acids taken from position 147 to position 170 of SEQ ID NO. 2.

SEQ ID NO. 1 is the sequence of bovine OPN (UniProtKB/Swiss-Prot Entry P31096) and SEQ ID NO. 2 is the sequence of human OPN (UniProtKB/Swiss-Prot Entry P10451).

The isolated, pharmaceutically active, molecule is also referred to as the “active molecute”.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), betabranched side chains (e.g., threonine, valine, isoleucine) and aromatic side chains (e.g., tyrosine, phenylalanine, tryptophan, histidine). Thus, a predicted nonessential amino acid residue is preferably replaced with another amino acid residue from the same side chain family.

The isolated, pharmaceutically active, molecule may e.g. comprise, or even consist of, an amino acid sequence of at least three consecutive amino acid residues from SEQ ID NO. 22, said amino acid sequence containing at most 15 consecutive amino acids taken from position 147 to position 170 of SEQ ID NO. 2.

In the context of the present invention, when a molecule or peptide fragment contains an amino acid sequence of a number of amino acid residues from a larger reference amino acid sequence, the amino acid sequence refers to a number of consecutive amino acids which are found in the same consecutive order in the larger reference amino acid sequence.

It should be noted that SEQ ID NO. 22 includes a number of possible conservative amino acid substitutions/variations at several positions in the sequence and that all combinations of these variations are encompassed by SEQ ID NO. 22.

In some embodiments of the invention, position 1 of SEQ ID NO. 22 is Gly.

In some embodiments of the invention, position 2 of SEQ ID NO. 22 is Asp.

In some embodiments of the invention, position 3 of SEQ ID NO. 22 is Ser.

In some embodiments of the invention, position 4 of SEQ ID NO. 22 is Val.

In some embodiments of the invention, position 5 of SEQ ID NO. 22 is Ala.

In some embodiments of the invention, position 7 of SEQ ID NO. 22 is Gly.

In some embodiments of the invention, position 8 of SEQ ID NO. 22 is Leu.

In some embodiments of the invention the isolated, pharmaceutically active, molecule comprises an amino acid sequence of at least three amino acid residues from:
position 153 to position 160 of SEQ ID NO. 1, or
position 160 to position 167 of SEQ ID NO. 2.

The isolated, pharmaceutically active, molecule may for example comprise an amino acid sequence of at least three amino acid residues from position 153 to position 160 of SEQ ID NO. 1.

Alternatively, the isolated, pharmaceutically active, molecule may for example comprise an amino acid sequence of at least three amino acid residues from position 160 to position 167 of SEQ ID NO. 2.

In some embodiments of the invention, the active molecule has a molecular weight of at most 5 kg/mol. For example, the active molecule may have a molecular weight of at most 4 kg/mol. The active molecule may e.g. have a molecular weight of at most 3 kg/mol. Alternatively, the active molecule may have a molecular weight of at most 2 kg/mol.

Even smaller active molecules may be useful, thus the active molecule may e.g. have a molecular weight of at most 1.5 kg/mol. For example, the active molecule may have a molecular weight of at most 1.2 kg/mol. Alternatively, the active molecule may have a molecular weight of at most 1.0 kg/mol.

The active molecule may have a molecular weight of at most 0.8 kg/mol. For example, the active molecule may have a molecular weight of at most 0.6 kg/mol. The active molecule may e.g. have a molecular weight of at most 0.5 kg/mol. Alternatively, the active molecule may have a molecular weight of at most 0.4 kg/mol.

The active molecule as such may be an OPN-derived peptide fragment as defined herein or a peptide fragment containing one or more conservative amino acid substitutions relative to the OPN-derived peptide fragment.

The amino acid sequence of the active molecule may be an OPN-derived peptide fragment as defined herein, i.e. having the same sequence and potentially the same modifications as an OPN-derived peptide fragment.

The one or more of the side groups, the N-terminal amino group and/or the C-terminal carboxylic acid group may have been modified, e.g. by phosphorylation or glycosylation.

In some embodiments of the invention, the C-terminal amino acid residue of the amino acid sequence of the active molecule is unmodified. For example, the C-terminal carboxylic acid group (in protonated or de-protonated form) of the amino acid sequence of the active molecules may be unmodified.

In some embodiments of the invention, the N-terminal amino acid residue of the amino acid sequence of the active molecule is unmodified. For example, the N-terminal amino group (in protonated or de-protonated form) of the amino acid sequence of the active molecules may be unmodified.

In some preferred embodiments of the invention, the amino acid sequence of the active molecule is selected from the group consisting of SEQ ID NO 7, 20 and 21.

For example, the amino acid sequence of the active molecule may be SEQ ID NO 7. Alternatively, the amino acid sequence of the active molecule may be SEQ ID NO 20. The amino acid sequence of the active molecule may e.g. be SEQ ID NO 21.

In some preferred embodiments of the invention the at least one amino acid residue of the amino acid sequence is phosphorylated. For example, the serine residue of SEQ ID NO 7 or SEQ ID NO 20 may be a phosphorylated serine residue.

In other preferred embodiments of the invention none of the amino acid residues of the amino acid sequence are phosphorylated.

The active molecule may contain, or even consist of, a linear sequence of amino acids.

Alternatively, the active molecule may contain a ring structure involving at least two amino acid residues. The C-terminal amino acid residue may for example be linked to the N-terminal amino acid residue, either directly or by means of a linker group.

In some embodiments of the invention, the amino acid sequence of the active molecule contains an unmodified amino group at the N-terminal amino acid residue and:
a) a neighbouring Asp having an unmodified side group, or
b) a phosphorylated serine residue two positions away from N-terminal amino acid.

In some embodiments of the invention, the amino acid sequence of the active molecule contains an Asp having an unmodified side group next to a phosphorylated serine residue.

In some preferred embodiments of the invention the isolated, pharmaceutically active, molecule comprises at most 30 amino acids. For example, the isolated, pharmaceutically active, molecule may comprise at most 25 amino acids. Preferably, the isolated, pharmaceutically active, molecule comprises at most 20 amino acids. The isolated, pharmaceutically active, molecule may for example comprise at most 15 amino acids.

In other preferred embodiments of the invention the isolated, pharmaceutically active, molecule comprises at most 10 amino acids. For example, the isolated, pharmaceutically active, molecule may comprise at most 8 amino acids. Preferably, the isolated, pharmaceutically active, molecule comprises at most 5 amino acids. The isolated, pharmaceutically active, molecule may for example comprise at most 4 amino acids, such as e.g. 3 amino acids.

The isolated, pharmaceutically active, molecule may e.g. comprise 3-30 amino acids. In some preferred embodiments of the invention the isolated, pharmaceutically active, molecule comprises 3-25 amino acids. For example, the isolated, pharmaceutically active, molecule may comprise 3-20 amino acids, such as e.g. 4-20 amino acids. Alternatively, the isolated, pharmaceutically active, molecule may comprise 3-15 amino acids, such as e.g. 5-15 amino acids. The isolated, pharmaceutically active, molecule may for example comprise 3-10 amino acids, such as e.g. 5-10 amino acids.

Yet an aspect of the invention pertains to an isolated, pharmaceutically active, OPN-derived peptide fragment comprising a sequence of at least two amino acid residues from position 140 to position 170 of SEQ ID NO. 1 or from position 147 to position 177 of SEQ ID NO. 2.

In the context of the present invention, the term “peptide fragment” pertains to a sequence of at least two directly linked amino acid residues, which sequence is a fragment of a larger peptide.

In the context of the present invention, the term “OPN-derived peptide fragment” pertains to peptide fragment found in OPN, but not necessarily obtained directly from OPN. An OPN-derived peptide fragment may for example be obtained by chemical synthesis, by fermentation or by protease treatment of OPN followed by purification of the peptide fragment. The OPN-derived peptide fragment may e.g. consist of the amino acid residues as such or it may furthermore contain various glycolylations and/or phosphorylations in the appropriate side groups or C- or N-terminus of the peptide fragment.

In the context of the present invention the term “amino acid” used in the context of a protein, a peptide, or a peptide fragment, means an amino acid residue which forms part or the protein, peptide, or peptide fragment, and not a free amino acid.

In the context of the present invention, a “sequence of at least X amino acid residues” taken from a larger reference sequence, such as the reference sequence from position 140 to position 170 of SEQ ID NO. 1, is a sequence of at least X directly linked amino acid residues, which sequence is also found within the reference sequence.

In the context of the present invention, an OPN-derived peptide fragment or a molecule is deemed pharmaceutically active if it is capable of reducing the growth of a cancer tumor in a mammal subject. The pharmaceutical activity of an OPN-derived peptide fragment or an active molecule may e.g. be tested using the procedure outlined in Example 1 (oral administration) or Example 3B (administration by intra-peritoneal injection).

In the context of the present invention, an “isolated OPN-derived peptide fragment” or an “isolated, pharmaceutically active, molecule” has been isolated to a purity of at least 10% (w/w), preferably at 25% (w/w), and even more preferably at least 40% (w/w).

For example, an “isolated OPN-derived peptide fragment” or an “isolated, pharmaceutically active, molecule” may have been isolated to a purity of at least 60% (w/w). Preferably, an “isolated OPN-derived peptide fragment” or an “isolated, pharmaceutically active, molecule” has been isolated to a purity of at least at 80% (w/w), and more preferably to at least 90% (w/w). Even more preferably, the “isolated OPN-derived peptide fragment” or the “isolated, pharmaceutically active, molecule” may be substantially pure, i.e. isolated to a purity of at least 95% (w/w), such as e.g. approx. 100% (w/w).

In some preferred embodiments of the invention, the isolated, pharmaceutically active, OPN-derived peptide fragment comprises a sequence of at least two amino acid residues from position 140 to position 170 of SEQ ID NO. 1.

In other preferred embodiments of the invention, the isolated, pharmaceutically active, OPN-derived peptide fragment comprises a sequence of at least two amino acid residues from position 147 to position 177 of SEQ ID NO. 2.

When the phrase “from position X to position Y of SEQ ID NO. Z” is used herein the amino acids of position X and position Y are included in the range. For example, the amino acid sequence from position 147-151 of SEQ ID NO. 1 is the penta-peptide SerAla-Asn-Asp-Gly.

The isolated, pharmaceutically active, OPN-derived peptide fragment may for example comprise, or even consist of, a sequence of at least 3 amino acid residues from position 140 to position 170 of SEQ ID NO. 1, such as a sequence of at least 4 amino acid residues or even a sequence of at least 5 amino acid residues from position 140 to position 170 of SEQ ID NO. 1.

Alternatively, the isolated, pharmaceutically active, OPN-derived peptide fragment may comprise, or even consist of, a sequence of at least 2 amino acid residues from position 152 to position 161 of SEQ ID NO. 1, such as a sequence of at least 4 amino acid residues or even a sequence of at least 5 amino acid residues from position 152 to position 161 of SEQ ID NO. 1.

For example, the isolated, pharmaceutically active, OPN-derived peptide fragment may comprise, or even consist of, a sequence of at least 3 amino acid residues from position 153 to position 160 of SEQ ID NO. 1, such as a sequence of at least 4 amino acid residues or even a sequence of at least 5 amino acid residues from position 152 to position 161 of SEQ ID NO. 1.

In some preferred embodiments of the invention the isolated, pharmaceutically active, OPN-derived peptide fragment comprises, or even consists of, a sequence of 2 to 25 amino acid residues from position 140 to position 170 of SEQ ID NO. 1. The isolated, pharmaceutically active, OPN-derived peptide fragment may for example comprise, or even consist of, a sequence of 3 to 15 amino acid residues from position 140 to position 170 of SEQ ID NO. 1. Alternatively, the isolated, pharmaceutically active, OPN-derived peptide fragment may comprise, or even consist of, a sequence of 4 to 6 amino acid residues from position 140 to position 170 of SEQ ID NO. 1.

In other preferred embodiments of the invention the isolated, pharmaceutically active, OPN-derived peptide fragment comprises, or even consists of, a sequence of 2 to 12 amino acid residues from position 152 to position 161 of SEQ ID NO. 1. The isolated, pharmaceutically active, OPN-derived peptide fragment may for example comprise, or even consist of, a sequence of 3 to 10 amino acid residues from position 152 to position 161 of SEQ ID NO. 1. Alternatively, the isolated, pharmaceutically active, OPN-derived peptide fragment may comprise, or even consist of, a sequence of 4 to 6 amino acid residues from position 152 to position 161 of SEQ ID NO. 1.

The isolated, pharmaceutically active, OPN-derived peptide fragment may e.g. comprise, or even consist of, a sequence of 3 to 10 amino acid residues from position 153 to position 160 of SEQ ID NO. 1. Alternatively, the isolated, pharmaceutically active, OPN-derived peptide fragment may comprise, or even consist of, a sequence of 4 to 6 amino acid residues from position 153 to position 160 of SEQ ID NO. 1.

The isolated, pharmaceutically active, OPN-derived peptide fragment may for example comprise, or even consist of, a sequence of at least 3 amino acid residues from position 147 to position 177 of SEQ ID NO. 2, such as a sequence of at least 4 amino acid residues or even a sequence of at least 5 amino acid residues from position 147 to position 177 of SEQ ID NO. 2.

Alternatively, the isolated, pharmaceutically active, OPN-derived peptide fragment may comprise, or even consist of, a sequence of at least 2 amino acid residues from position 159 to position 168 of SEQ ID NO. 2, such as a sequence of at least 4 amino acid residues or even a sequence of at least 5 amino acid residues from position 159 to position 168 of SEQ ID NO. 2.

In some preferred embodiments of the invention the isolated, pharmaceutically active, OPN-derived peptide fragment comprises, or even consists of, a sequence of 2 to 25 amino acid residues from position 147 to position 177 of SEQ ID NO. 2. The isolated, pharmaceutically active, OPN-derived peptide fragment may for example comprise, or even consist of, a sequence of 3 to 15 amino acid residues from position 147 to position 177 of SEQ ID NO. 2. Alternatively, the isolated, pharmaceutically active, OPN-derived peptide fragment may comprise, or even consist of, a sequence of 4 to 6 amino acid residues from position 147 to position 177 of SEQ ID NO. 2.

In other preferred embodiments of the invention the isolated, pharmaceutically active, OPN-derived peptide fragment comprises, or even consists of, a sequence of 2 to 12 amino acid residues from position 159 to position 168 of SEQ ID NO. 2. The isolated, pharmaceutically active, OPN-derived peptide fragment may for example comprise, or even consist of, a sequence of 3 to 10 amino acid residues from position 159 to position 168 of SEQ ID NO. 2. Alternatively, the isolated, pharmaceutically active, OPN-derived peptide fragment may comprise, or even consist of, a sequence of 4 to 6 amino acid residues from position 159 to position 168 of SEQ ID NO. 2.

In some preferred embodiments of the invention, the isolated, pharmaceutically active, OPN-derived peptide fragment comprises, or even consists of, an RGD motif, i.e. the amino acid sequence Arg-Gly-Asp.

In some embodiments of the invention, the isolated, pharmaceutically active, OPN-derived peptide fragment comprises, or even consists of, a peptide having a sequence identity of at least 80% relative to an amino acid sequence selected from the group consisting of SEQ ID NO. 3, SEQ ID NO. 4, SEQ ID NO. 5, and SEQ ID NO. 6.

In the context of the present invention, the term “sequence identity” relates to a quantitative measure of the degree of identity between two amino acid sequences or between two nucleic acid sequences, preferably of equal length. If the two sequences to be compared are not of equal length, they must be aligned to the best possible fit. The sequence identity can be calculated as

((N_{ref}−N_{dif})*100)/(N_{ref}),

wherein N_{dif} is the total number of non-identical residues in the two sequences when aligned, and wherein N_{ref} is the number of residues of the reference sequences. Hence, the DNA sequence AGTCAGTC will have a sequence identity of 75% with the sequence AATCAATC (N_{dif}=2 and N_{ref}=8). A gap is counted as non-identity of the specific residue(s), i.e. the DNA sequence AGTGTC will have a sequence identity of 75% with the DNA sequence AGTCAGTC (Ndif=2 and Nref=8). Sequence identity can for example be calculated using appropriate BLAST-programs, such as the BLASTp-algorithm provided by National Center for Biotechnology Information (NCBI), USA.

The isolated, pharmaceutically active, OPN-derived peptide fragment may comprise, or even consist of, a peptide having a sequence identity of at least 80% relative to an amino acid sequence selected from the group consisting of SEQ ID NO. 7, SEQ ID NO. 8, SEQ ID NO. 9, and SEQ ID NO. 10.

In some preferred embodiments of the invention the OPN-derived peptide fragment comprises, or even consists of, a peptide having a sequence identity of at least 80% relative to an amino acid sequence selected from the group consisting of SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, and SEQ ID NO. 20.

Alternatively, the OPN-derived peptide fragment may comprise, or even consist of, a peptide having a sequence identity of at least 80% relative to an amino acid sequence selected from the group consisting of SEQ ID NO. 21 and SEQ ID NO. 22.

In other preferred embodiments of the invention, the isolated, pharmaceutically active, OPN-derived peptide fragment comprises, or even consists of, an amino acid sequence selected from the group consisting of SEQ ID NO. 3, SEQ ID NO. 4, SEQ ID NO. 5, and SEQ ID NO. 6.

In further preferred embodiments of the invention, the isolated, pharmaceutically active, OPN-derived peptide fragment comprises, or even consists of, an amino acid sequence selected from the group consisting of SEQ ID NO. 7, SEQ ID NO. 8, SEQ ID NO. 9, and SEQ ID NO. 10.

In additional preferred embodiments of the invention, the isolated, pharmaceutically active, OPN-derived peptide fragment comprises, or even consists of, an amino acid sequence selected from the group consisting of SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, and SEQ ID NO. 20.

Alternatively, the OPN-derived peptide fragment may comprise, or even consist of, a peptide having an amino acid sequence selected from the group consisting of SEQ ID NO. 21 and SEQ ID NO. 22.

The isolated, pharmaceutically active, OPN-derived peptide fragment may for example comprise, or even consist of, the amino acid sequence of SEQ ID NO. 3. Alternatively, the isolated, pharmaceutically active, OPN-derived peptide fragment may comprise, or even consist of, the amino acid sequence of SEQ ID NO. 4. For example, the isolated, pharmaceutically active, OPN-derived peptide fragment may comprise, or even consist of, the amino acid sequence of SEQ ID NO. 5.

The isolated, pharmaceutically active, OPN-derived peptide fragment may for example comprise, or even consist of, the amino acid sequence of SEQ ID NO. 6. For example, the isolated, pharmaceutically active, OPN-derived peptide fragment may comprise, or even consist of, the amino acid sequence of SEQ ID NO. 7.

The isolated, pharmaceutically active, OPN-derived peptide fragment may for example comprise, or even consist of, the amino acid sequence of SEQ ID NO. 8. Alternatively, the isolated, pharmaceutically active, OPN-derived peptide fragment may comprise, or even consist of, the amino acid sequence of SEQ ID NO. 9. For example, the isolated, pharmaceutically active, OPN-derived peptide fragment may comprise, or even consist of, the amino acid sequence of SEQ ID NO. 10.

The isolated, pharmaceutically active, OPN-derived peptide fragment may for example comprise, or even consist of, the amino acid sequence of SEQ ID NO. 11. Alternatively, the isolated, pharmaceutically active, OPN-derived peptide fragment may comprise, or even consist of, the amino acid sequence of SEQ ID NO. 12. For example, the isolated, pharmaceutically active, OPN-derived peptide fragment may comprise, or even consist of, the amino acid sequence of SEQ ID NO. 13.

The isolated, pharmaceutically active, OPN-derived peptide fragment may for example comprise, or even consist of, the amino acid sequence of SEQ ID NO. 14. Alternatively, the isolated, pharmaceutically active, OPN-derived peptide fragment may comprise, or even consist of, the amino acid sequence of SEQ ID NO. 15. For example, the isolated, pharmaceutically active, OPN-derived peptide fragment may comprise, or even consist of, the amino acid sequence of SEQ ID NO. 16.

The isolated, pharmaceutically active, OPN-derived peptide fragment may for example comprise, or even consist of, the amino acid sequence of SEQ ID NO. 17. Alternatively, the isolated, pharmaceutically active, OPN-derived peptide fragment may comprise, or even consist of, the amino acid sequence of SEQ ID NO. 18. For example, the isolated, pharmaceutically active, OPN-derived peptide fragment may comprise, or even consist of, the amino acid sequence of SEQ ID NO. 19. The isolated, pharmaceutically active, OPN-derived peptide fragment may e.g. comprise, or even consist of, the amino acid sequence of SEQ ID NO. 20.

The isolated, pharmaceutically active, OPN-derived peptide fragment may for example comprise, or even consist of, the amino acid sequence of SEQ ID NO. 21. Alternatively, the isolated, pharmaceutically active, OPN-derived peptide fragment may comprise, or even consist of, the amino acid sequence of SEQ ID NO. 22.

Other useful isolated, pharmaceutically active, OPN-derived peptide fragments are mentioned in Tables 1, 2, 3, and 4.

In some embodiments of the invention, the isolated, pharmaceutically active, OPN-derived peptide fragment is a di-peptide. The isolated, pharmaceutically active, OPN-derived peptide fragment may for example be a di-peptide from Table 1. Alternatively, the isolated, pharmaceutically active, OPN-derived peptide fragment may be a di-peptide from Table 2.

In other embodiments of the invention, the isolated, pharmaceutically active, OPN-derived peptide fragment is a tri-peptide. The isolated, pharmaceutically active, OPN-derived peptide fragment may for example be a tri-peptide from Table 1. Alternatively, the isolated, pharmaceutically active, OPN-derived peptide fragment may be a tri-peptide from Table 2.

TABLE 1
Useful di- and tri-peptide fragments. AAS No. = Amino
acid sequence No.

| | |
|---|---|
| Di-peptides | Tri-peptides |
| Position in | Position in |
| SEQ ID NO. 1 | SEQ ID NO. 1 |

| AAS No. | From | To | AAS No. | From | To |
|---|---|---|---|---|---|
| 1 | 140 | 141 | 31 | 140 | 142 |
| 2 | 141 | 142 | 32 | 141 | 143 |
| 3 | 142 | 143 | 33 | 142 | 144 |
| 4 | 143 | 144 | 34 | 143 | 145 |
| 5 | 144 | 145 | 35 | 144 | 146 |
| 6 | 145 | 146 | 36 | 145 | 147 |
| 7 | 146 | 147 | 37 | 146 | 148 |
| 8 | 147 | 148 | 38 | 147 | 149 |
| 9 | 148 | 149 | 39 | 148 | 150 |
| 10 | 149 | 150 | 40 | 149 | 151 |
| 11 | 150 | 151 | 41 | 150 | 152 |
| 12 | 151 | 152 | 42 | 151 | 153 |
| 13 | 152 | 153 | 43 | 152 | 154 |
| 14 | 153 | 154 | 44 | 153 | 155 |
| 15 | 154 | 155 | 45 | 154 | 156 |
| 16 | 155 | 156 | 46 | 155 | 157 |
| 17 | 156 | 157 | 47 | 156 | 158 |
| 18 | 157 | 158 | 48 | 157 | 159 |
| 19 | 158 | 159 | 49 | 158 | 160 |
| 20 | 159 | 160 | 50 | 159 | 161 |
| 21 | 160 | 161 | 51 | 160 | 162 |
| 22 | 161 | 162 | 52 | 161 | 163 |
| 23 | 162 | 163 | 53 | 162 | 164 |
| 24 | 163 | 164 | 54 | 163 | 165 |
| 25 | 164 | 165 | 55 | 164 | 166 |
| 26 | 165 | 166 | 56 | 165 | 167 |
| 27 | 166 | 167 | 57 | 166 | 168 |
| 28 | 167 | 168 | 58 | 167 | 169 |
| 29 | 168 | 169 | 59 | 168 | 170 |
| 30 | 169 | 170 | 60 | 169 | 171 |

TABLE 2
Useful di- and tri-peptide fragments. AAS No. = Amino
acid sequence No.

| | |
|---|---|
| Di-peptides | Tri-peptides |
| Position in | Position in |
| SEQ ID NO. 2 | SEQ ID NO. 2 |

| AAS No. | From | To | AAS No. | From | To |
|---|---|---|---|---|---|
| 61 | 147 | 148 | 91 | 147 | 149 |
| 62 | 148 | 149 | 92 | 148 | 150 |
| 63 | 149 | 150 | 93 | 149 | 151 |
| 64 | 150 | 151 | 94 | 150 | 152 |
| 65 | 151 | 152 | 95 | 151 | 153 |
| 66 | 152 | 153 | 96 | 152 | 154 |
| 67 | 153 | 154 | 97 | 153 | 155 |
| 68 | 154 | 155 | 98 | 154 | 156 |
| 69 | 155 | 156 | 99 | 155 | 157 |
| 70 | 156 | 157 | 100 | 156 | 158 |
| 71 | 157 | 158 | 101 | 157 | 159 |
| 72 | 158 | 159 | 102 | 158 | 160 |
| 73 | 159 | 160 | 103 | 159 | 161 |
| 74 | 160 | 161 | 104 | 160 | 162 |
| 75 | 161 | 162 | 105 | 161 | 163 |
| 76 | 162 | 163 | 106 | 162 | 164 |
| 77 | 163 | 164 | 107 | 163 | 165 |
| 78 | 164 | 165 | 108 | 164 | 166 |
| 79 | 165 | 166 | 109 | 165 | 167 |
| 80 | 166 | 167 | 110 | 166 | 168 |
| 81 | 167 | 168 | 111 | 167 | 169 |
| 82 | 168 | 169 | 112 | 168 | 170 |
| 83 | 169 | 170 | 113 | 169 | 171 |
| 84 | 170 | 171 | 114 | 170 | 172 |
| 85 | 171 | 172 | 115 | 171 | 173 |
| 86 | 172 | 173 | 116 | 172 | 174 |
| 87 | 173 | 174 | 117 | 173 | 175 |
| 88 | 174 | 175 | 118 | 174 | 176 |
| 89 | 175 | 176 | 119 | 175 | 177 |
| 90 | 176 | 177 | 120 | 176 | 178 |

In further embodiments of the invention, the isolated, pharmaceutically active, OPN-derived peptide fragment is a tetra-peptide. The isolated, pharmaceutically active, OPN-derived peptide fragment may for example be a tetra-peptide from Table 3. Alternatively, the isolated, pharmaceutically active, OPN-derived peptide fragment may be a tetra-peptide from Table 4.

TABLE 3
Useful tetra- and penta-peptide fragments. AAS No. = Amino
acid sequence No.

| | |
|---|---|
| Tetra-peptides | Penta-peptides |
| Position in | Position in |
| SEQ ID NO. 1 | SEQ ID NO. 1 |

| AAS No. | From | To | AAS No. | From | To |
|---|---|---|---|---|---|
| 121 | 140 | 143 | 151 | 140 | 144 |
| 122 | 141 | 144 | 152 | 141 | 145 |
| 123 | 142 | 145 | 153 | 142 | 146 |
| 124 | 143 | 146 | 154 | 143 | 147 |
| 125 | 144 | 147 | 155 | 144 | 148 |
| 126 | 145 | 148 | 156 | 145 | 149 |
| 127 | 146 | 149 | 157 | 146 | 150 |
| 128 | 147 | 150 | 158 | 147 | 151 |
| 129 | 148 | 151 | 159 | 148 | 152 |
| 130 | 149 | 152 | 160 | 149 | 153 |
| 131 | 150 | 153 | 161 | 150 | 154 |
| 132 | 151 | 154 | 162 | 151 | 155 |
| 133 | 152 | 155 | 163 | 152 | 156 |
| 134 | 153 | 156 | 164 | 153 | 157 |
| 135 | 154 | 157 | 165 | 154 | 158 |
| 136 | 155 | 158 | 166 | 155 | 159 |
| 137 | 156 | 159 | 167 | 156 | 160 |
| 138 | 157 | 160 | 168 | 157 | 161 |
| 139 | 158 | 161 | 169 | 158 | 162 |
| 140 | 159 | 162 | 170 | 159 | 163 |
| 141 | 160 | 163 | 171 | 160 | 164 |
| 142 | 161 | 164 | 172 | 161 | 165 |
| 143 | 162 | 165 | 173 | 162 | 166 |
| 144 | 163 | 166 | 174 | 163 | 167 |
| 145 | 164 | 167 | 175 | 164 | 168 |
| 146 | 165 | 168 | 176 | 165 | 169 |
| 147 | 166 | 169 | 177 | 166 | 170 |
| 148 | 167 | 170 | 178 | 167 | 171 |
| 149 | 168 | 171 | 179 | 168 | 172 |
| 150 | 169 | 172 | 180 | 169 | 173 |

TABLE 4
Useful tetra- and penta-peptide fragments. AAS No. = Amino
acid sequence No.

| | |
|---|---|
| Tetra-peptides | Penta-peptides |
| Position in | Position in |
| SEQ ID NO. 2 | SEQ ID NO. 2 |

| AAS No. | From | To | AAS No. | From | To |
|---|---|---|---|---|---|
| 181 | 147 | 150 | 211 | 147 | 151 |
| 182 | 148 | 151 | 212 | 148 | 152 |
| 183 | 149 | 152 | 213 | 149 | 153 |
| 184 | 150 | 153 | 214 | 150 | 154 |
| 185 | 151 | 154 | 215 | 151 | 155 |
| 186 | 152 | 155 | 216 | 152 | 156 |
| 187 | 153 | 156 | 217 | 153 | 157 |
| 188 | 154 | 157 | 218 | 154 | 158 |
| 189 | 155 | 158 | 219 | 155 | 159 |
| 190 | 156 | 159 | 220 | 156 | 160 |
| 191 | 157 | 160 | 221 | 157 | 161 |
| 192 | 158 | 161 | 222 | 158 | 162 |
| 193 | 159 | 162 | 223 | 159 | 163 |
| 194 | 160 | 163 | 224 | 160 | 164 |
| 195 | 161 | 164 | 225 | 161 | 165 |
| 196 | 162 | 165 | 226 | 162 | 166 |
| 197 | 163 | 166 | 227 | 163 | 167 |
| 198 | 164 | 167 | 228 | 164 | 168 |
| 199 | 165 | 168 | 229 | 165 | 169 |
| 200 | 166 | 169 | 230 | 166 | 170 |
| 201 | 167 | 170 | 231 | 167 | 171 |
| 202 | 168 | 171 | 232 | 168 | 172 |
| 203 | 169 | 172 | 233 | 169 | 173 |
| 204 | 170 | 173 | 234 | 170 | 174 |
| 205 | 171 | 174 | 235 | 171 | 175 |
| 206 | 172 | 175 | 236 | 172 | 176 |
| 207 | 173 | 176 | 237 | 173 | 177 |
| 208 | 174 | 177 | 238 | 174 | 178 |
| 209 | 175 | 178 | 239 | 175 | 179 |
| 210 | 176 | 179 | 240 | 176 | 180 |

In yet other embodiments of the invention, the isolated, pharmaceutically active, OPN-derived peptide fragment is a penta-peptide. The isolated, pharmaceutically active, OPN-derived peptide fragment may for example be a penta-peptide from Table 3. Alternatively, the isolated, pharmaceutically active, OPN-derived peptide fragment may be a penta-peptide from Table 4.

In some preferred embodiments of the invention the isolated, pharmaceutically active, OPN-derived peptide fragment comprises at most 30 amino acids. For example, the isolated, pharmaceutically active, OPN-derived peptide fragment may comprise at most 25 amino acids. Preferably, the isolated, pharmaceutically active, OPN-derived peptide fragment comprises at most 20 amino acids. The isolated, pharmaceutically active, OPN-derived peptide fragment may for example comprise at most 15 amino acids.

In other preferred embodiments of the invention the isolated, pharmaceutically active, OPN-derived peptide fragment comprises at most 10 amino acids. For example, the isolated, pharmaceutically active, OPN-derived peptide fragment may comprise at most 8 amino acids. Preferably, the isolated, pharmaceutically active, OPN-derived peptide fragment comprises at most 5 amino acids. The isolated, pharmaceutically active, OPN-derived peptide fragment may for example comprise at most 4 amino acids, such as at most 3 amino acids.

The isolated, pharmaceutically active, OPN-derived peptide fragment may for example comprise 2-30 amino acids. In some preferred embodiments of the invention the isolated, pharmaceutically active, OPN-derived peptide fragment comprises 3-30, such as e.g. 3-25 amino acids. For example, the isolated, pharmaceutically active, OPN-derived peptide fragment may comprise 3-20 amino acids, such as e.g. 4-20 amino acids. Alternatively, the isolated, pharmaceutically active, OPN-derived peptide fragment may comprise 3-15 amino acids, such as e.g. 5-15 amino acids. The isolated, pharmaceutically active, OPN-derived peptide fragment may for example comprise 3-10 amino acids, such as e.g. 5-10 amino acids.

In some preferred embodiments of the invention the isolated, pharmaceutically active, OPN-derived peptide fragment comprises at least one glycosylated amino acid. For example, the isolated, pharmaceutically active, OPN-derived peptide fragment may contain a glycosylated threonine residue. The glycosyl group may for example be a sialyl group. Alternatively the glycosyl group may be a galactosyl group, a glycosyl group, or an n-actyl-galactosaminyl group.

In some preferred embodiments of the invention the isolated, pharmaceutically active, OPN-derived peptide fragment comprises at least one phosphorylated amino acid. For example, the isolated, pharmaceutically active, OPN-derived peptide fragment may contain a phosphorylated serine, threonine, tyrosine, histidine or arginine or lysine residue.

Yet an aspect of the invention pertains to an isolated, pharmaceutically active, OPN-derived peptide fragment or an isolated, pharmaceutically active, molecule as defined herein for use as a medicament. For example, an aspect of the invention pertains to an active agent comprising, or even consisting of, an isolated, pharmaceutically active, OPN-derived peptide fragment or an isolated, pharmaceutically active, molecule as defined herein for use as a medicament.

A further aspect of the invention pertains to an active agent comprising, or even consisting of, one or more isolated, pharmaceutically active, OPN-derived peptide fragment(s) or one or more isolated, pharmaceutically active, molecules as defined herein for use in the treatment or prevention of cancer involving at least one cancer tumor.

A cancer tumor comprises several tumor cells (neoplastic cells), which are characterized by abnormal cell growth or replication. In some instances, abnormal cell growth (for example of a localized region of cells) results in formation of a tumor cell mass (neoplasm, solid lesion). Abnormal cell growth is relative to the growth of cells which do not form a tumor cell mass. Abnormal cells may exhibit an abnormal (for example increased) rate of division as compared to normal cells. In some embodiments, tumor cells are pre-malignant or malignant. Malignant tumor cells may be referred to as cancer cells and may have the ability to metastasize or spread to neighboring tissues or other locations in the body and grow there as new tumors.

It is particularly preferred that the at least one cancer tumor has an elevated level of OPN.

Thus, in some preferred embodiments of the invention the active agent is for use in the treatment or prevention of cancer involving at least one cancer tumor, which cancer tumor has an elevated level of OPN.

In additional preferred embodiments of the invention the tumor is capable of inducing an elevated concentration of OPN in the plasma of the subject having the tumor.

In certain embodiments, the tumor cells are of epithelial origin. Epithelial cells reside in one or more layers which cover the entire surface of the body and which line most of the hollow structures of the body, excluding the blood vessels, lymph vessels, and the heart interior, which are lined with endothelium, and the chest and abdominal cavities which are lined with mesothelium.

Epithelial tumors include benign and premalignant epithelial tumors, such as breast fibroadenoma and colon adenoma, and malignant epithelial tumors. Malignant epithelial tumors include primary tumors, also referred to as carcinomas, and secondary tumors, also referred to as metastases of epithelial origin. Carcinomas include acinar carcinoma, acinous carcinoma, alveolar adenocarcinoma (also called adenocystic carcinoma, adenomyoepithelioma, cribriform carcinoma and cylindroma), carcinoma adenomatosum, adenocarcinoma, carcinoma of adrenal cortex, alveolar carcinoma, alveolar cell carcinoma (also called bronchiolar carcinoma, alveolar cell tumor and pulmonary adenomatosis), basal cell carcinoma, carcinoma basocellulare (also called basaloma, or basiloma, and hair matrix carcinoma), basaloid carcinoma, basosquamous cell carcinoma, breast carcinoma, bronchioalveolar carcinoma, bronchiolar carcinoma, bronchogenic carcinoma, cerebriform carcinoma, cholangiocellular carcinoma (also called cholangioma and cholangiocarcinoma), chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliale adenoides, carcinoma exulcere, carcinoma fibrosum, gelatiniform carcinoma, gelatinous carcinoma, giant cell carcinoma, gigantocellulare, glandular carcinoma, granulosa cell carcinoma, hair-matrix carcinoma, hematoid carcinoma, hepatocellular carcinoma (also called hepatoma, malignant hepatoma and hepatocarcinoma), Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lenticular carcinoma, carcinoma lenticulare, lipomatous carcinoma, lymphoepithelial carcinoma, carcinoma mastitoides, carcinoma medullare, medullary carcinoma, carcinoma melanodes, melanotic carcinoma, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, carcinoma nigrum, oat cell carcinoma, carcinoma ossificans, osteoid carcinoma, ovarian carcinoma, papillary carcinoma, periportal carcinoma, preinvasive carcinoma, prostate carcinoma, renal cell carcinoma of kidney (also called adenocarcinoma of kidney and hypernephoroid carcinoma), reserve cell carcinoma, carcinoma sarcomatodes, scheinderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma vilosum.

In some preferred embodiments of the invention, the cancer tumor is a fibrosarcoma.

In other embodiments, the tumor cells are of mesenchymal origin, for example, tumor cells forming a sarcoma. Sarcomas are rare mesenchymal neoplasms that arise in bone and soft tissues. Different types of sarcomas include liposarcomas (including myxoid liposarcomas and pleiomorphic liposarcomas), leiomyosarcomas, rhabdomyosarcomas, malignant peripheral nerve sheath tumors (also called malignant schwannomas, neurofibrosarcomas, or neurogenic sarcomas), Ewing's tumors (including Ewing's sarcoma of bone, extraskeletal [not bone] Ewing's sarcoma, and primitive neuroectodermal tumor [PNET]), synovial sarcoma, angiosarcomas, hemangiosarcomas, lymphangiosarcomas, Kaposi's sarcoma, hemangioendothelioma, fibrosarcoma, desmoid tumor (also called aggressive fibromatosis), dermatofibrosarcoma protuberans (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) (also known as GI stromal sarcoma), osteosarcoma (also known as osteogenic sarcoma)-skeletal and extraskeletal, and chondrosarcoma.

In some preferred embodiments of the invention, the cancer tumor is an adenocarcinoma or a breast carcinoma.

In some embodiments, the tumor cells are of melanocytic origin. Melanomas are tumors arising from the melanocytic system of the skin and other organs. Examples of melanoma include lentigo maligna melanoma, superficial spreading melanoma, nodular melanoma, and acral lentiginous melanoma.

In still other embodiments, the tumor cells include those found in biliary tract cancer, endometrial cancer, esophageal cancer, gastric cancer, intraepithelial neoplasms, including Bowen's disease and Paget's disease, liver cancer, oral cancer, including squamous cell carcinoma, sarcomas, including fibrosarcoma and osteosarcoma, skin cancer, including melanoma, Kaposi's sarcoma, testicular cancer, including germinal tumors (seminoma, non-seminoma (teratomas, choriocarcinomas)), stromal tumors and germ cell tumors, thyroid cancer, including thyroid adenocarcinoma and medullar carcinoma, and renal cancer, including adenocarcinoma and Wilms' tumor.

In particular embodiments, the tumor cells may originate in bone, muscle or connective tissue. The tumor cells may be found in primary tumors (for example sarcomas) of bone and connective tissue.

In other embodiments, the tumor cells may be metastatic. In some embodiments, the metastatic tumors are of epithelial origin. Carcinomas may metastasize to bone, as has been observed with breast cancer, and liver, as is sometimes the case with colon cancer. The methods provided herein are directed to suppressing growth or replication of metastatic tumors irrespective of the site of the metastasis and/or the site of the primary tumor.

The active agent may e.g. comprise one or more isolated, pharmaceutically active, OPN-derived peptide fragment(s). However, in some preferred embodiments of the invention, the active agent consists of one or more isolated, pharmaceutically active, OPN-derived peptide fragment(s).

The active agent may e.g. comprise, or even consist of, an OPN-derived peptide fragment having the sequence of SEQ ID NO. 7. Alternatively, the active agent may e.g. comprise, or even consist of, an OPN-derived peptide fragment having the sequence of SEQ ID NO. 20. Alternatively, the active agent may e.g. comprise, or even consist of, an OPN-derived peptide fragment having the sequence of SEQ ID NO. 21.

The active agent may also contain a mixture of different peptide fragments. For example, the active agent may comprise, or even consist of, at least two peptides having a sequence selected from the group consisting of SEQ ID NO. 7, SEQ ID NO. 20, and SEQ ID NO. 21.

The active agent may e.g. contain a mixture of three peptides having the sequences SEQ ID NO. 7, SEQ ID NO. 20, and SEQ ID NO. 21.

The serine residue of SEQ ID NO. 20 is preferably a phosphorylated serine residue. The serine residue of SEQ ID NO. 7 may also be a phosphorylated serine residue.

Alternatively or additionally, the active agent may comprise one or more isolated, pharmaceutically active, molecules. However, in some preferred embodiments of the invention, the active agent consists of one or more isolated, pharmaceutically active, molecules.

The active agent may e.g. comprise, or even consist of, an active molecule wherein the amino acid sequence has the sequence of SEQ ID NO. 7. Alternatively, the active agent may e.g. comprise, or even consist of, an active molecule wherein the amino acid sequence has the sequence of SEQ ID NO. 20. For example, the active agent may e.g. comprise, or even consist of, an active molecule wherein the amino acid sequence has the sequence of SEQ ID NO. 21.

In some preferred embodiments of the invention, the active agent comprises, or even consist of, an active molecule which is a peptide having the sequence of SEQ ID NO. 7. Alternatively, the active agent may e.g. comprise, or even consist of, an active molecule which is a peptide having the sequence of SEQ ID NO. 20. For example, the active agent may e.g. comprise, or even consist of, an active molecule which is a peptide having the sequence of SEQ ID NO. 21.

The active agent may also contain a mixture of different active molecules. For example, the active agent may comprise, or even consist of, at least two active molecules containing an amino acid sequence selected from the group consisting of SEQ ID NO. 7, SEQ ID NO. 20, and SEQ ID NO. 21.

The active agent may e.g. contain a mixture of three different active molecules, the first active molecule containing an amino acid sequence having the sequences of SEQ ID NO. 7, the second active molecule containing an amino acid sequence having the sequence of SEQ ID NO. 20, and the third active molecule containing an amino acid sequence having the sequence of SEQ ID NO. 21.

When the active agent contains a single type of OPN-derived peptide fragment or active molecule, the OPN-derived peptide fragment or active molecule is typically present in an amount in the range of 10-100% (w/w) relative to the total weight of the active agent.

When the active agent contains two types of OPN-derived peptide fragments or active molecules, the OPN-derived peptide fragments or active molecules are typically each present in an amount in the range of 1-90% (w/w) relative to the total weight of the active agent.

For example, the active agent may comprise, or even consist of, two active molecules containing an amino acid sequence selected from the group consisting of SEQ ID NO. 7, SEQ ID NO. 20, and SEQ ID NO. 21, which two active molecules are each present in an amount in the range of 1-90% (w/w) relative to the total weight of the active agent. The two active molecules may e.g. each be present in an amount in the range of 20-70% (w/w) relative to the total weight of the active agent.

When the active agent contains three types of OPN-derived peptide fragments or active molecules, the OPN-derived peptide fragments or active molecules are typically each present in an amount in the range of 1-80% (w/w) relative to the total weight of the active agent.

For example, the active agent may comprise, or even consist of, three active molecules each containing a sequence selected from the group consisting of SEQ ID NO. 7, SEQ ID NO. 20, and SEQ ID NO. 21, which three active molecules are each present in an amount in the range of 1-90% (w/w) relative to the total weight of the active agent. The two active molecules may e.g. each be present in an amount in the range of 20-70% (w/w) relative to the total weight of the active agent.

The one or more isolated, pharmaceutically active, OPN-derived peptide fragment(s) may be isolated from natural source of such compounds. Alternatively, the one or more isolated, pharmaceutically active, OPN-derived peptide fragment(s) may be prepared by fermentation or by chemical synthesis.

In some preferred embodiments of the invention the active agent comprises, or even consists of, one OPN-derived peptide fragment. However, the active agent may also contain more OPN-derived peptide fragments, such as at least two OPN-derived peptide fragments or at least three OPN-derived peptide fragments.

In some embodiments of the invention, the active agent is a hydrolysate of OPN formed by protease digestion, e.g. a hydrolysate of OPN purified from bovine milk.

In certain embodiments, the active agent is derived from milk, and in particular embodiments, the milk is bovine milk. In other embodiments, the active agent is derived from other domesticated milk producing mammals, including goat, sheep, buffalo, llama, and camel.

In some embodiments, the active agent is purified with respect to the one or more isolated, pharmaceutically active, OPN-derived peptide fragment(s). In some embodiments, the active agent is at least about 50% to about 60%, at least about 60% to about 70%, or at least about 70% to about 80% pure. In some embodiments, it is at least about 80% to about 90% pure, while in other embodiments, the active agent is at least about 90% to about 95% pure, or more. In certain embodiments, the purified active agent is at least about 95% pure, such as 95%, 96%, 97%, 98%, 99%, or 99.5% pure, or more.

For example, the active agent may contain a total amount of active OPN-derived peptide fragments and active molecules of at least 50% (w/w) relative to the weight of the active agent, preferably at least 60% (w/w), and more preferably at least 70% (w/w). The active agent may e.g. contain a total amount of active OPN-derived peptide fragments and active molecules of at least 80% (w/w) relative to the weight of the active agent, preferably at least 90% (w/w), and more preferably at least 95% (w/w). Thus, in some embodiments, the active agent is at least about 95% pure, such as 95%, 96%, 97%, 98%, 99%, or 99.5% pure, or more.

In some embodiments of the invention, the active agent contains a total amount of active molecules as defined herein of at least 50% (w/w) relative to the weight of the active agent, preferably at least 60% (w/w), and more preferably at least 70% (w/w). The active agent may e.g. contain a total amount of active molecules of at least 80% (w/w) relative to the weight of the active agent, preferably at least 90% (w/w), and more preferably at least 95% (w/w). Thus, in some embodiments, the active agent is at least about 95% pure with respect to the one or more active molecules, such as 95%, 96%, 97%, 98%, 99%, or 99.5% pure, or more.

The active agent may be administered in several ways.

In some preferred embodiments of the invention the treatment or prevention is by oral administration. Oral administration may for example involve sublingual administration and/or buccal administration. Alternatively, or additionally, oral administration may involve that the active agent enters the gastrointestinal system.

Alternatively, the active agent may be administered by parenterally, e.g. by injection or infusion. Thus, in some preferred embodiments of the invention the treatment or prevention may for example be by intravenous (IV) administration of the active agent. In other preferred embodiments of the invention the treatment or prevention may for example be by intramuscular or subcutaneous administration, such as intramuscular or subcutaneous injection. In other preferred embodiments of the invention the treatment or prevention may for example be by intra-peritoneal administration, such as intra-peritoneal injection.

In further embodiments of the invention the treatment or prevention may for example be by nasal administration.

In some embodiments of the invention the treatment or prevention is for suppressing, and/or reducing, tumor cell growth or replication. For example, the treatment or prevention may be for preventing tumor cell replication. The treatment or prevention may e.g. be for preventing tumor cell growth.

In some preferred embodiments of the invention medical uses and methods provided herein are directed to suppressing, and/or reducing, growth or replication of tumor cells regardless of their site of origin.

The treatment or prevention may also be for suppressing, and/or reducing, cancer tumor growth.

In some preferred embodiments of the invention, the treatment is for preventing growth of a cancer tumor. For example, the treatment may be for preventing growth and/or replication of the cancer cells of a cancer tumor.

In some embodiments of the invention the treatment or prevention is for reducing the risk of metastasis in a subject having a cancer involving at least one cancer tumor. For example, the treatment or prevention may be for preventing metastasis in a subject having a cancer involving at least one cancer tumor.

The treatment or prevention may for example prevent tumor cell growth or replication.

In some preferred embodiments of the invention, the subject to be treated is a human subject.

In some preferred embodiments of the invention, the subject to be treated has a cancer involving at least one cancer tumor having an elevated level of osteopontin.

In the context of the present invention, a cancer tumor has an elevated level of OPN if the level of OPN in the cancer tumor is at least 1 nanogram/microgram protein. The level of OPN in a cancer tumor is determined according to Example 4. In some embodiments of the invention a cancer tumor is deemed to have an elevated level of OPN if the level of OPN in the cancer tumor is at least 5 nanogram/microgram protein. For example, a cancer tumor may be deemed to have an elevated level of OPN if the level of OPN in the cancer tumor is at least 10 nanogram/microgram protein. In other embodiments of the invention a cancer tumor is deemed to have an elevated level of OPN if the level of OPN in the cancer tumor is at least 20 nanogram/microgram protein. For example, a cancer tumor may be deemed to have an elevated level of OPN if the level of OPN in the cancer tumor is at least 50 nanogram/microgram protein.

Alternatively, an elevated level of osteopontin in a cancer tumor can be determined by immunohistochemistry essentially as described for human tumor samples (Tuck 1998). 4-6 micron sections of formalin fixed, paraffin embedded tumor tissues are rehydrated and subjected to antigen retrieval by boiling for 12 minutes in 0.01 M NaCitrate, pH 6.0. After blocking in 5% goat serum, anti-osteopontin antibody (R&D #AF808 or Santa Cruz Biotechnologies mAK2A1) will be diluted according to the manufacturer's instructions and incubated with the tissue sections for 1 hour. Secondary antibody incubation and detection is performed using the Vector ABC Elite kit comprising biotinylated antigoat antibody and avidin-biotin complex (Vector cat #PK-6105); detection is achieved by staining with diaminobenzidine (DAB, included in kit). Extent and intensity of staining is determined microscopically and graded according to a semi-quantitative system described in (Tuck 1998). Tumor samples with a score greater than 4 will be considered having an elevated level of OPN.

In other preferred embodiments of the invention the subject having the cancer tumor has an elevated concentration of OPN in the plasma derived from its blood.

In the context of the present invention, a subject has an elevated concentration of OPN in its plasma if the plasma concentration of OPN is at least 80 nanogram/mL. The concentration of OPN in plasma derived from the blood of a subject is determined according to Example 5. In some embodiments of the invention a subject has an elevated concentration of OPN in its plasma if the plasma concentration of OPN is at least 100 nanogram/mL. For example, a subject may have an elevated concentration of OPN in its plasma if the plasma concentration of OPN is at least 120 nanogram/mL. In other embodiments of the invention a subject has an elevated concentration of OPN in its plasma if the plasma concentration of OPN is at least 140 nanogram/mL. For example, a subject may have an elevated concentration of OPN in its plasma if the plasma concentration of OPN is at least 180 nanogram/mL.

In some preferred embodiments of the invention, the subject to which the active agent is, or is to be, administered has an increased risk of developing a cancer involving at least one cancer tumor.

In the context of the present invention, a subject is deemed to have an increased risk of developing a cancer involving at least one cancer tumor if the subject's lifetime risk of developing a cancer tumor is at least 20% higher than that calculated for persons from the general population matched for gender, age and ethnicity.

An example of increased risk is a 55 year old woman with a first degree relative with breast cancer: this woman's lifetime risk of developing breast cancer is 36% higher than the general population (see e.g. the Breast Cancer Risk Assessment Tool on www.cancer.gov/bcrisktool/which is an interactive tool designed by scientists at the National Cancer Institute (NCI)).

The increased risk may be caused by hereditary or environmental circumstances or by the life style of the subject.

In some embodiments of the invention, the increased risk is caused by an environmental circumstance. The subject may for example have been exposed to a significant amount of radioactive radiation or to a significant amount of a carcinogenic substance.

In other embodiments of the invention, the increased risk is caused by the life style of the subject. The subject may for example be a tobacco-smoker, or an ex-tobacco-smoker.

In still other embodiments of the invention, the increased risk is caused by heritage from a parent of the subject. The subject may for example have at least one firstdegree relative, e.g. a mother, father, sister or brother, who has breast cancer, lung cancer, ovary cancer or colon cancer.

The subject having an increased risk of developing cancer may for example have a genetic profile which is associated with an increased risk of developing a cancer involving at least one cancer tumor.

In the context of the present invention, the term “genetic profile” relates both to the genes which the subject has inherited from its parents and to gene mutations which have been caused by environmental circumstances.

In some embodiments of the invention, the genetic profile comprises at least one inherited gene which is associated with an increased risk of developing a cancer involving at least one cancer tumor. An example of such a gene is the BRCA1 gene or the BRCA2 gene. See for example Nelson 2005.

In the context of the present invention, a genetic profile is deemed to be associated with an increased risk of developing a cancer involving at least one cancer tumor if the lifetime risk of developing the cancer is at least 20% higher for carriers of the genetic profile than for non-carriers.

In some preferred embodiments of the invention, the subject which is, or is to be, treated is also treated with another type of anti-cancer treatment. Examples of such other kinds of anti-cancer treatment are for example chemotherapy, chemoprevention, targeted therapy, bone-marrow transplant, radiation treatment, surgery, or a combination thereof.

In some preferred embodiments of the invention the active agent is administered in a daily dosage in the range of about 0.05 mg/kg of body weight to about 5 g/kg of body weight of the subject treated.

The rate of tumor cell growth and, consequently, the overall size of a tumor cell mass may be reduced and in certain embodiments statistically significantly reduced in a subject, if the active agent is administered (for example orally) to the subject. Suppression of tumor cell growth resulting from a subject having received an effective amount of the active agent is relative to the rate of tumor cell growth of that same tumor prior to the subject receiving the active agent, or relative to tumor cell growth of a comparable tumor (comparable in initial size of the mass and cell type) in a subject not having been exposed to an effective amount of the active agent. Tumor cell growth may refer to the rate of cell division or replication, or the overall size (for example volume or circumference) of the tumor cell mass. Methods of measuring a tumor cell mass are well known in the art. For example, see Tomayko 1989, incorporated herein by reference.

In the context of the present invention the size of a cancer tumor refers to the volume of the tumor. The rate of growth of a cancer tumor refers to the volumetric growth of the tumor per time unit. The volume of a cancer tumor may be determined by conventional imaging techniques such as MRI scanner or ultrasonic imaging.

In certain embodiments, the rate of growth or size of a tumor cell mass may be reduced by at least about 5% to about 10%, as compared to the rate of growth or size of a similar tumor not exposed to a therapeutically effective amount of the active agent. In other embodiments, the tumor cell mass may be reduced by at least about 10% to about 15%, at least about 15% to about 20%, at least about 20% to about 25%, at least about 25% to about 30%, at least about 30% to about 35%, at least about 35% to about 40%, at least about 40% to about 45%, at least about 45% to about 50%, at least about 50% to about 55%, at least about 55% to about 60%, at least about 60% to about 65%, at least about 65% to about 70%, at least about 70% to about 75%, at least about 75% to about 80%, at least about 80% to about 85%, or at least about 85% to about 90%, or more. In yet other embodiments, the tumor cell mass is reduced by at least about 50%. In particular embodiments, the tumor cell mass is reduced by at least about 75%.

Alternatively, the rate of growth of a tumor cell mass may be reduced about 5% to about 100%, as compared to the rate of growth or size of a similar tumor not exposed to a therapeutically effective amount of the active agent. For example, the rate of growth of a tumor cell mass may be reduced about 20% to about 95%. Preferably, the rate of growth of a tumor cell mass is reduced about 40% to about 100%. Even more preferably, the rate of growth of a tumor cell mass is reduced about 60% to about 100%.

The active agent, as described herein, may be administered to a subject in an amount effective to suppress growth or replication of tumor cells. In certain embodiments, the active agent may be administered at a concentration of about 0.05 mg/ml to about 1 mg/ml. In some embodiments, it may be administered at a concentration of about 0.05 mg/ml to about 0.1 mg/ml, about 0.1 mg/ml to about 0.15 mg/ml, about 0.15 mg/ml to about 0.2 mg/ml, about 0.25 mg/ml to about 0.3 mg/ml, about 0.3 mg/ml to about 0.35 mg/ml, about 0.35 mg/ml to about 0.4 mg/ml, about 0.4 mg/ml to about 0.45 mg/ml, about 0.45 mg/ml to about 0.5 mg/ml, about 0.55 mg/ml to about 0.6 mg/ml, about 0.6 mg/ml to about 0.65 mg/ml, about 0.65 mg/ml to about 0.7 mg/ml, about 0.7 mg/ml to about 0.75 mg/ml, about 0.75 mg/ml to about 0.8 mg/ml, about 0.85 mg/ml to about 0.9 mg/ml, about 0.9 mg/ml to about 0.95 mg/ml, or about 0.95 mg/ml to about 1 mg/ml. In one embodiment, the active agent may be administered at a concentration of 0.03 mg/ml. In another embodiment, the active agent may be administered at a concentration of 0.12 mg/ml. In yet another embodiment, the active agent may be administered at a concentration of 0.3 mg/ml.

In particular embodiments, the active agent may be administered at a concentration of about 1 mg/ml to about 0.1 g/ml. In some embodiments, it may be administered at a concentration of about 1 mg/ml to about 5 mg/ml, about 5 mg/ml to about 10 mg/ml, about 10 mg/ml to about 15 mg/ml, about 15 mg/ml to about 20 mg/ml, about 20 mg/ml to about 25 mg/ml, about 25 mg/ml to about 30 mg/ml, about 30 mg/ml to about 35 mg/ml, about 35 mg/ml to about 40 mg/ml, about 40 mg/ml to about 45 mg/ml, about 45 mg/ml to about 50 mg/ml, about 50 mg/ml to about 55 mg/ml, about 55 mg/ml to about 60 mg/ml, about 60 mg/ml to about 65 mg/ml, about 65 mg/ml to about 70 mg/ml, about 70 mg/ml to about 75 mg/ml, about 75 mg/ml to about 80 mg/ml, about 80 mg/ml to about 85 mg/ml, about 85 mg/ml to about 90 mg/ml, about 90 mg/ml to about 95 mg/ml, or about 95 mg/ml to about 0.1 g/ml.

In some embodiments, the active agent may be administered at a concentration of about 0.1 g/ml to about 1 g/ml. In other embodiments, it may be administered at a concentration of about 0.1 g/ml to about 0.15 g/ml, about 0.15 g/ml to about 0.2 g/ml, about 0.2 g/ml to about 0.25 g/ml, about 0.25 g/ml to about 0.3 g/ml, about 0.3 g/ml to about 0.35 g/ml, about 0.35 g/ml to about 0.4 g/ml, about 0.4 g/ml to about 0.45 g/ml, about 0.45 g/ml to about 0.5 g/ml, about 0.5 g/ml to about 0.55 g/ml, about 0.55 g/ml to about 0.6 g/ml, about 0.6 g/ml to about 0.65 g/ml, about 0.65 g/ml to about 0.7 g/ml, about 0.7 g/ml to about 0.75 g/ml, about 0.75 g/ml to about 0.8 g/ml, about 0.8 g/ml to about 0.85 g/ml, about 0.85 g/ml to about 0.9 g/ml, about 0.9 g/ml to about 0.95 g/ml, or about 0.95 g/ml to about 1 g/ml.

In certain embodiments, the active agent may be administered in a daily dosage of about 0.05 mg/kg of body weight to about 1 mg/kg of body weight. In the context of the present invention, the unit “mg/kg” or “g/kg” mentioned in the context of a daily dosage of the active agent, relates to the daily amount of the active agent in mg or g per kg body weight of the subject to be treated.

In some embodiments, it may be administered in a daily dosage of about 0.05 mg/kg to about 0.1 mg/kg, about 0.1 mg/kg to about 0.15 mg/kg, about 0.15 mg/kg to about 0.2 mg/kg, about 0.25 mg/kg to about 0.3 mg/kg, about 0.3 mg/kg to about 0.35 mg/kg, about 0.35 mg/kg to about 0.4 mg/kg, about 0.4 mg/kg to about 0.45 mg/kg, about 0.45 mg/kg to about 0.5 mg/kg, about 0.55 mg/kg to about 0.6 mg/kg, about 0.6 mg/kg to about 0.65 mg/kg, about 0.65 mg/kg to about 0.7 mg/kg, about 0.7 mg/kg to about 0.75 mg/kg, about 0.75 mg/kg to about 0.8 mg/kg, about 0.85 mg/kg to about 0.9 mg/kg, about 0.9 mg/kg to about 0.95 mg/kg, or about 0.95 mg/kg to about 1 mg/kg.

In other embodiments, the active agent may be administered in a daily dosage of about 1 mg/kg to about 0.1 g/kg of bodyweight. In other additional embodiments, it may be administered in a daily dosage of about 1 mg/kg to about 5 mg/kg, about 5 mg/kg to about 10 mg/kg, about 10 mg/kg to about 15 mg/kg, about 15 mg/kg to about 20 mg/kg, about 20 mg/kg to about 25 mg/kg, about 25 mg/kg to about 30 mg/kg, about 30 mg/kg to about 35 mg/kg, about 35 mg/kg to about 40 mg/kg, about 40 mg/kg to about 45 mg/kg, about 45 mg/kg to about 50 mg/kg, about 50 mg/kg to about 55 mg/kg, about 55 mg/kg to about 60 mg/kg, about 60 mg/kg to about 65 mg/kg, about 65 mg/kg to about 70 mg/kg, about 70 mg/kg to about 75 mg/kg, about 75 mg/kg to about 80 mg/kg, about 80 mg/kg to about 85 mg/kg, about 85 mg/kg to about 90 mg/kg, about 90 mg/kg to about 95 mg/kg, or about 95 mg/kg to about 0.1 g/kg.

In some embodiments of the invention, the active agent may be administered in a daily dosage of about 0.1 g/kg to about 1 g/kg of bodyweight. In certain embodiments, it may be administered in a daily dosage of about 0.1 g/kg to about 0.15 g/kg, about 0.15 g/kg to about 0.2 g/kg, about 0.2 g/kg to about 0.25 g/kg, about 0.25 g/kg to about 0.3 g/kg, about 0.3 g/kg to about 0.35 g/kg, about 0.35 g/kg to about 0.4 g/kg, about 0.4 g/kg to about 0.45 g/kg, about 0.45 g/kg to about 0.5 g/kg, about 0.5 g/kg to about 0.55 g/kg, about 0.55 g/kg to about 0.6 g/kg, about 0.6 g/kg to about 0.65 g/kg, about 0.65 g/kg to about 0.7 g/kg, about 0.7 g/kg to about 0.75 g/kg, about 0.75 g/kg to about 0.8 g/kg, about 0.8 g/kg to about 0.85 g/kg, about 0.85 g/kg to about 0.9 g/kg, about 0.9 g/kg to about 0.95 g/kg, or about 0.95 g/kg to about 1 g/kg.

In particular embodiments, the active agent may be administered in a daily dosage of about 1 g/kg to about 5 g/kg of bodyweight. In some embodiments, it may be administered in a daily dosage of about 1 g/kg to about 1.5 g/kg, about 1.5 g/kg to about 2 g/kg, about 2 g/kg to about 2.5 g/kg, about 2.5 g/kg to about 3 g/kg, about 3 g/kg to about 3.5 g/kg, about 3.5 g/kg to about 4 g/kg, about 4 g/kg to about 4.5 g/kg, about 4.5 g/kg to about 5 g/kg.

In certain embodiments, the active agent is administered in a daily dosage in the range of about 0.05 mg/kg to 5 g/kg. For example, the active agent may be administered in a daily dosage in the range of about 1 mg/kg to 0.5 g/kg. Alternatively, the active agent may be administered in a daily dosage in the range of about 0.005 g/kg to 0.2 g/kg. The active agent may e.g. be administered in a daily dosage in the range of about 0.01 g/kg to 0.1 g/kg.

In other embodiments, the active agent is administered in a daily dosage in the range of about 1 mg/kg body weight to 300 mg/kg body weight. For example, the active agent may be administered in a daily dosage in the range of about 5 mg/kg body weight to 250 mg/kg body weight. Alternatively, the active agent may be administered in a daily dosage in the range of about 10 mg/kg body weight to 200 mg/kg body weight. The active agent may e.g. be administered in a daily dosage in the range of about 30 mg/kg body weight to 150 mg/kg body weight.

Yet an aspect of the invention relates to the use of an active agent comprising, or even consisting of, the one or more isolated, pharmaceutically active, OPN-derived peptide fragment(s) or the one or more isolated, pharmaceutically active, molecules as defined herein in the manufacture of a medicament for use in the treatment or prevention of cancer involving at least one cancer tumor.

Yet an aspect of the invention pertains to a method of treating or preventing cancer, the method comprising: administering to a subject having cancer, or to a subject being at risk of getting cancer, an amount of an active agent comprising, or even consisting of, the one or more isolated, pharmaceutically active, OPN-derived peptide fragment(s) or the one or more isolated, pharmaceutically active, molecules effective to treat or prevent said cancer, and where said cancer involves at least one cancer tumor.

In some preferred embodiments of the invention the method comprises administering to a subject having the cancer, or to a subject being at risk of getting the cancer, an amount of an active agent comprising, or even consisting of, the one or more isolated, pharmaceutically active, OPN-derived peptide fragment(s) or the one or more isolated, pharmaceutically active, molecules as defined herein, effective to suppress tumor cell growth or replication.

The method of treatment may for example be a method of reducing the risk of or preventing metastasis in a subject having a cancer involving at least one cancer tumor, for example a cancer tumor having an elevated level of osteopontin, the method comprising: administering to the subject an amount of an active agent comprising, or even consisting of, the one or more isolated, pharmaceutically active, OPN-derived peptide fragment(s) or the one or more isolated, pharmaceutically active, molecules effective to reduce the risk of, or even prevent, metastasis.

An aspect of the invention pertains to methods of suppressing tumor cell growth or replication in a subject by administering to the subject the active agent, or a pharmaceutical composition comprising the active agent, in an amount effective to suppress tumor cell growth or replication. Such an effective amount may be referred to herein as a therapeutically effective amount.

The term “effective amount” or “therapeutically effective amount” as used herein means that the amount of the active agent administered to the subject directly or comprised within a pharmaceutical composition or nutritional supplement is of sufficient quantity to cause the mentioned effect, for example suppression of tumor growth and/or suppression of tumor cell growth or replication in the subject. The effective amounts may be determined empirically by persons of skill in the art, for example medical practitioners. Factors, such as age, height and weight, of a subject may be considered when for example determining the amount of the active agent effective to suppress tumor growth and/or tumor cell growth or replication.

Another aspect of the invention pertains to a pharmaceutical composition comprising:
an active agent comprising, or even consisting of, the one or more isolated, pharmaceutically active, OPN-derived peptide fragment(s) or the one or more isolated, pharmaceutically active, molecules as defined herein, and
a pharmaceutically acceptable carrier.

The active agent is preferably present in the pharmaceutical compositions mentioned herein in a pharmaceutically effective amount.

In some preferred embodiments of the invention the pharmaceutical composition comprises the active agent in an amount in the range of 0.01-90% (w/w). For example, the pharmaceutical composition may comprise the active agent in an amount in the range of 0.1-80% (w/w). Alternatively, the pharmaceutical composition may comprise the active agent in an amount in the range of 1-70% (w/w).

In some embodiments of the invention the pharmaceutical composition comprises the active agent in an amount in the range of 5-60% (w/w). For example, the pharmaceutical composition may comprise the active agent in an amount in the range of 10-50% (w/w). Alternatively, the pharmaceutical composition may comprise the active agent in an amount in the range of 0.1-20% (w/w).

In addition to the active agent, the pharmaceutical composition may furthermore comprise one or more additional therapeutic agent(s). The one or more additional therapeutic agent(s) is preferably an anti-cancer agent.

Examples of targeted therapeutic agents include small molecules, such as imatinib mesylate (GLEEVEC®, also known as STI-571), Gefitinib (IRESSA®, also known as ZD1839), Erlotinib (Tarceva®), bortezomib (VELCADE®), Bcl-2 inhibitors (for example obatoclax mesylate, ABT-263, and gossypol), PARP inhibitors (for example iniparib, olaparib), Janus kinase inhibitors, PI3K inhibitors, Apatinib (a selective VEGF receptor 2 inhibitor), and salinomycin. Examples of targeted therapeutic agents also include monoclonal antibodies, such as Rituximab (marketed as MABTHERA® or RITUXAN®), Trastuzumab (HERCEPTIN®), Cetuximab (ERBITUX®), Bevacizumab (AVASTATIN®). Examples also include antibody-drug conjugates.

Examples of chemotherapeutic agents include alkylating agents (for example cisplatin, carboplatin, oxaliplatin, mechlorethamine, cyclophosphamide, chlorambucil, ifosfamide), anti-metabolites (for example purines (such as azathioprine, mercaptopurine) and pyrimidines), plant alkaloids and terpenoids (for example vinca alkaloids such as vincristine, vinblastine, vinorelbine, and vindesine, podophyllotoxins, and taxanes), topoisomerase inhibitors (for example irinotecan, topotecan, amsacrine, etoposide, etoposide phosphate, and teniposide), and cytotoxic inhibitors (for example actinomycin such as actinomycin D, anthracyclines such as doxorubicin (L01DB01), daunorubicin (L01DB02), valrubicin, idarubicin, epirubicin (L01DB03), and other cytotoxic antibiotics such as bleomycin (L01DC01), plicamycin (L01DC02), and mitomycin (L01DC03)).

Another example of a useful therapeutic agent is an integrin blocking agent, e.g. an alpha, beta₃ integrin blocking agent such as cilengitide.

Examples of immunosuppressive agents include glucocorticoids, cytostatics (for example alkylating agents and antimetabolites such as folic acid analogues (for example methotrexate), purine analogues (for example azathioprine, mercaptopurine), pyrimidine analogues, and protein synthesis inhibitors), antibodies (for example polyclonal and monoclonal), drugs acting on immunophilins (for example ciclosporin, tacrolimus, sirolimus), and other drugs, including interferons, opioids, TNF binding proteins, mycophenolate, and small biological agents (for example fingolimod, myriocin).

Other aspects described herein include pharmaceutical compositions comprising the active agent and a pharmaceutically acceptable carrier, one or more compatible solid or liquid fillers, or one or more diluents or encapsulating agents appropriate for the administration to a human or other animal. A carrier (or other agents) should be sufficiently pure and sufficiently low-toxic in order to be regarded as appropriate for the administration to a subject being treated. A carrier may be inert or may have its own pharmaceutically favorable properties. An amount of the carrier used in combination with the active agent may be sufficient to improve delivery and effectiveness of the active agent (for example the active agent delivery to cells or active agent uptake by cells) and may be determined empirically by those of skill in the art.

Examples of additional carriers or other (inactive) agents that may be comprised within the pharmaceutical compositions described herein include sugars, such as lactose, glucose and saccharose; starches, such as corn starch and potato starch; cellulose and derivatives thereof, such as sodium carboxymethylcellulose, ethylcellulose and methylcellulose; powdered tragacanth gum; gelatine; talc; solid lubricants, such as stearinic acid and magnesium stearate; calcium sulfate, vegetable oils, such as peanut butter, cottonseed oil and corn oil; polyols, such as propylene glycol, glycerin, sorbitol, mannitol and polyethylene glycol; alginic acid; emulsifiers, such as TWEEN®; wetting agents, such as sodium laurylsulfate; dyes; correctives; pelletizing agents; stabilizers; antioxidants; preservatives; pyrogen-free water; isotonic physiological solution, glucose solution and phosphate-buffered solutions; sweeteners, such as glycerin, propylene glycol, sorbitol, saccharose); correctives; flavoring agents; dyes; and preservatives, such as methyl- or n-propyl-p-hydroxy benzoate, sorbic acid, methyl paraben, benzoate. Optional active agents which do not significantly affect the activity of the compound of the present invention may be added to the pharmaceutical composition. Such active agents include anticancer targeted therapeutics and chemotherapeutic agents, and immunosuppressive or immunostimulating agents.

In certain embodiments, the pharmaceutical compositions containing the active agent are formulated for mucosal and/or oral administration. The compositions may be administered orally, sublingually, or buccally in standard dosage forms containing conventional nontoxic pharmaceutically acceptable carriers, adjuvants and media. The terms “oral” or “orally” may encompass “sublingual” or “sublingually” or “buccal” or “buccally”.

Pharmaceutically acceptable carriers specific for mucosal and/or oral administration are well-known in the art and include one or more sugars, starches, cellulose and derivative thereof, malt, gelatin, talc, calcium sulfate, vegetable oils, synthetic oils, polyols, alginic acid, phosphate-buffered solutions, emulsifiers, isotonic physiological solutions, ethanol, glycerin, propylene glycol, polyethylene glycol, sugar solution, sorbitol and water. Such compositions may also contain a demulcent.

Forms suitable for oral administration include tablets or granules, hard or soft capsules, pastilles, troches, suspensions in water or oil, emulsions, dispersible powders or granules, or syrups or elixirs. Pharmaceutical compositions formulated for oral administration may be prepared according to any method known in the art for the preparation of such compositions.

Tablets typically contain conventional pharmaceutically compatible auxiliary agents, such as inert diluents, such as calcium carbonate, sodium carbonate, mannitol, lactose and cellulose; binders, such as starch, gelatin and saccharose; dispersing agents, such as starch, alginic acid and croscarmellose; lubricants, such as magnesium stearate, stearic acid and talc. Glidants, such as silicon dioxide, may be used to improve fluidity characteristics of a powder composition. For appearance, dyes such as FD&C dyes may be added. Sweeteners and correctives, such as aspartame, saccharine, menthol, peppermint and fruit flavors are useful as adjuvants for chewable tablets. Capsules (including sustained release and delayed release preparations) typically contain one or more solid diluents described above. The selection of carrier components often depends on secondary factors, such as flavor, price and storage stability.

Pharmaceutical compositions in the form of tablets or capsules may also be coated using the conventional methods, typically with a pH-dependent coating. Such dosage forms typically comprise one or more components from among cellulose acetate phthalate, polyvinyl acetate phthalate, hydroxypropylmethylcellulose phthalate, ethyl cellulose, Eudragit coatings, waxes and shellac and other materials.

Active agent preparations for oral administration may be formulated into hard gelatin capsules, wherein the active agent is mixed with an inert solid diluent, for example calcium carbonate, calcium phosphate and kaolin, or in the form of soft gelatin capsules, wherein the active agent is mixed with water or an oil medium, for example peanut butter, liquid paraffin or olive oil.

Aqueous suspensions may comprise the active agent in a mixture with excipients suitable for obtaining aqueous suspensions. These excipients may be suspending agents, for example, carboxymethylcellulose sodium, methylcellulose, hydropropylmethyl cellulose, sodium alginate, polyvinyl pyrrolidone, tragacanth gum and Arabic gum; dispersing or wetting agents; naturally-occurring phosphatides, for example, lecithin, or products of condensation of alkylenoxide with fatty acids, for example, polyoxyethylene stearate, or products of condensation of ethylene oxide with long-chain aliphatic alcohols, for example, with heptadecaethylene oxycetanol, or products of condensation of ethylene oxide with partial esters produced from fatty acids and hexitol, such as substituted polyoxyethylene sorbitol, or products of condensation of ethylene oxide with partial esters produced from fatty acids and hexitol anhydrides, for example, substituted polyoxyethylene sorbitan. Aqueous suspensions may also contain some preservatives, for example ethylor n-propyl-p-hydroxybenzoate.

Oil suspensions may be prepared by suspending the active agent in a vegetable oil, for example, peanut butter, olive oil, sesame oil and coconut oil, or in a mineral oil, such as liquid paraffin. Oil suspensions may contain a thickening agent, for example, bee wax, solid paraffin or cetyl alcohol. Some sweeteners, such as mentioned above, and correctives can be added to obtain pleasant oral preparations. These compositions may be preserved by adding an anti-oxidant, such as ascorbic acid.

When the active agent exhibits insufficient solubility, solubilization methods may be used. Such methods are known to ones skilled in this field of art and comprise the use of co-solvents, such as dimethylsulfoxide (DMSO), the use of surfactants, such as TWEEN®, or dissolving in an aqueous solution of sodium bicarbonate and other methods.

The pharmaceutical compositions described herein may also be in the form of oil-inwater emulsions. The oil phase may represent a vegetable oil, for example, olive oil or peanut butter, or a mineral oil, for example, liquid paraffin or mixtures thereof. The appropriate emulsifiers may be naturally occurring gums, for example, Arabic gum or tragacanth gum, naturally occurring phosphatides, for example, soybean lecithin, and esters or partial esters produced from fatty acids and hexitol anhydrides, for example, sorbitan monooleate, and products of condensation of said partial esters with ethylene oxide, for example, polyoxyethylene sorbitan monooleate.

Dispersing powders and granules suitable for preparing an aqueous suspension include the active agent in a mixture with a dispersing or wetting agent, suspending agent and one or more preservatives. The appropriate dispersing or wetting agents and suspending agents include agents already exemplified above.

In one embodiment, the active agent or pharmaceutical compositions comprising the active agent may be administered in a nasal dosage form (for example nasal spray). Such compositions typically contain one or more fillers, such as saccharose, sorbitol and mannitol, and binders, such as Arabic gum, microcrystal cellulose, carboxymethylcellulose and hydroxypropylmethylcellulose. Glidants, lubricants, sweeteners, dyes, antioxidants and correctives described above can also be incorporated.

Pharmaceutical compositions for inhalation may be formulated in a solution, suspension or emulsion, which may be administered in the form of a dry powder or in the form of an aerosol using a conventional propellant (for example, dichlorodifluoromethane and trichlorofluoromethane).

In some preferred embodiments of the invention, the pharmaceutical composition is formulated for oral, sublingual, buccal, or nasal administration.

In other embodiments of the invention, the pharmaceutical composition is formulated for intravenous administration, e.g. for injection.

In some embodiments of the invention, the pharmaceutical composition is in a dosage form, which contains 90%-110% (w/w) of the daily dosage for an adult subject. In other embodiments of the invention, the pharmaceutical composition is in a dosage form, which contains 45%-55% (w/w) of the daily dosage for an adult subject. In further embodiments of the invention, the pharmaceutical composition is in a dosage form, which contains 28%-38% (w/w) of the daily dosage for an adult subject.

In some embodiments of the invention, the pharmaceutical composition is in a dosage form, which dosage form contains the active agent in an amount of the active agent in the range of 0.1 mg-10 g per dosage form. For example, the oral dosage form may contain an amount of the active agent in the range of 1 mg-1 g per dosage form. Alternatively, the oral dosage form may contain an amount of the active agent in the range of 10 mg-800 mg per dosage form. The oral dosage form may e.g. contain an amount of the active agent in the range of 25 mg-500 mg per dosage form.

Yet an aspect of the invention pertains to a nutritional supplement comprising
a nutritionally effective amount of an active agent comprising, or even consisting of, the one or more isolated, pharmaceutically active, OPN-derived peptide fragment(s) or the one or more isolated, pharmaceutically active, molecules, and
one or more components selected from the group consisting of a carbohydrate source, a lipid source, and a protein source.

In some preferred embodiments of the invention the nutritional supplement comprises the active agent in an amount in the range of 0.01-90% (w/w). For example, the nutritional supplement may comprise the active agent in an amount in the range of 0.1-80% (w/w). Alternatively, the nutritional supplement may comprise the active agent in an amount in the range of 1-70% (w/w).

In some embodiments of the invention the nutritional supplement comprises the active agent in an amount in the range of 5-60% (w/w). For example, the nutritional supplement may comprise the active agent in an amount in the range of 10-50% (w/w). Alternatively, the nutritional supplement may comprise the active agent in an amount in the range of 0.1-20% (w/w).

In other embodiments of the invention the nutritional supplement comprises the active agent in an amount in the range of 0.001-5% (w/w). For example, the nutritional supplement may comprise the active agent in an amount in the range of 0.005-2% (w/w). Alternatively, the nutritional supplement may comprise the active agent in an amount in the range of 0.01-1% (w/w). The nutritional supplement may e.g. comprise the active agent in an amount in the range of 0.05-0.5% (w/w).

Nutritional supplements comprising the active agent can be pre-packaged in liquid or powdered form (for example canned or bottled liquid drink). In some embodiments, the powdered form is added to a food or beverage to provide additional nutrients. In certain embodiments, the nutritional beverages are formulated with, for example, fruit, vegetables, yogurt, milk, and/or ice cream. In some embodiments, the nutritional supplements are blended to a smoothie consistency. In particular embodiments, the nutritional beverages are fortified with, for example, protein, vitamins, minerals, antioxidants, probiotics, and/or prebiotics. In certain embodiments, the nutritional beverages are lactose-free and/or gluten-free. In some embodiments, the nutritional supplements are organic. Examples of pediatric nutritional beverages include PEDIASURE®, PEDIASMART®, and RESOURCE® Just For Kids. Examples of adult nutritional beverages include ENSURE®, BOOST®, NESTLE® CARNATION® INSTANT BREAKFAST®, GLUCERNA®, GLYTROL®, NUTREN®, and PEPTAMEN®. Nutritional supplements also include milk, both soymilk and cow's milk (for example whole, semi-skim or low-fat, skim or non-fat (for example Cravendale), lactose-free (for example LACTOFREE®)).

The amount of the active agent comprised within the nutritional supplements described herein may be the same or similar to those amounts described above for the pharmaceutical compositions comprising the active agent, but may be lesser or greater amounts also. In particular embodiments, the amount of the active agent in the nutritional supplement is about 0.05 mg/ml to about 1 g/ml. In some embodiments, the subject may be a mammal. In some embodiment the subject may be murine, canine, feline, ovine, bovine, porcine, or equine, while in other embodiments the subject is human.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,”“comprising,” or “having,” “containing,” “involving,” and variations thereof herein, is meant to encompass the items listed thereafter and equivalents thereof as well as additional items.

Each of the foregoing patents, patent applications and references are hereby incorporated by reference, particularly for the teaching referenced herein.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### EXAMPLES

### Example 1

### Oral Administration of Osteopontin to 129B6F1 Tumor-Bearing Mice

A bovine OPN preparation (Lacprodan®, Arla Foods Ingredients, Viby, Denmark) was dissolved in deionized water, filtered, and administered at final concentrations of 0.03, 0.12, or 0.3 mg/ml to 129B6F1 tumor-bearing mice starting either on the day of tumor cell inoculation (Day 0) or five days later (Day 5). The mice were initially inoculated with 5×10⁴ mycoplasma-free 275-3-2 murine ras-transformed fibroblast cells (see Wu 2000). Tumor size was measured with calipers and monitored until the tumors reached 20% of the body weight of the animals, at which time the mice were sacrificed and tissue/plasma samples collected. Statistical significance was calculated using one-way ANOVA with Bonferroni post-test.

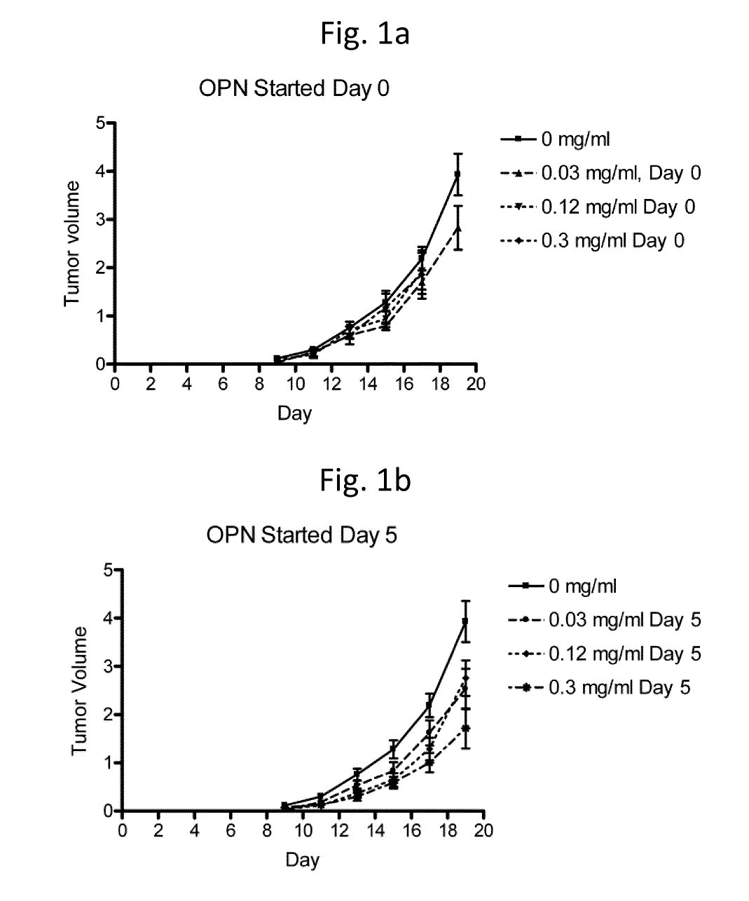

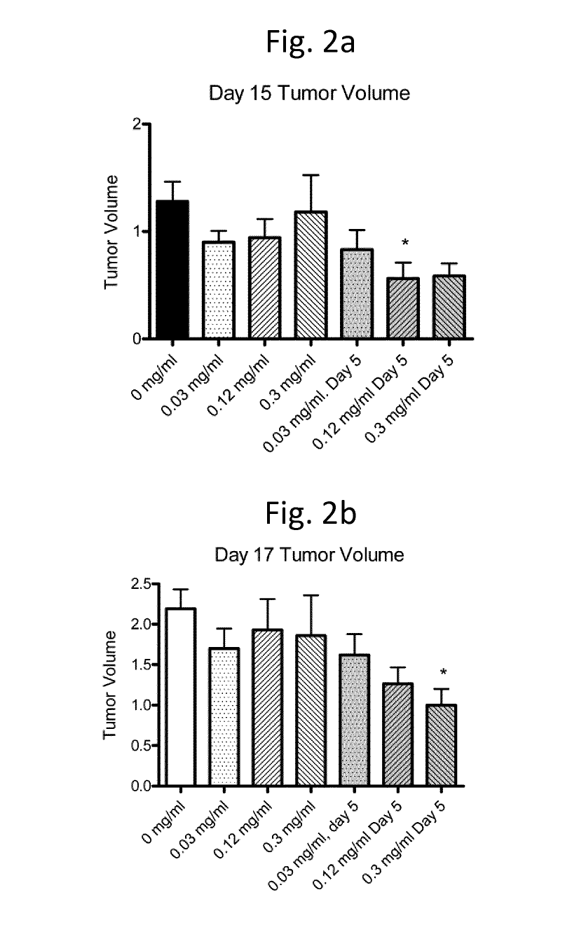

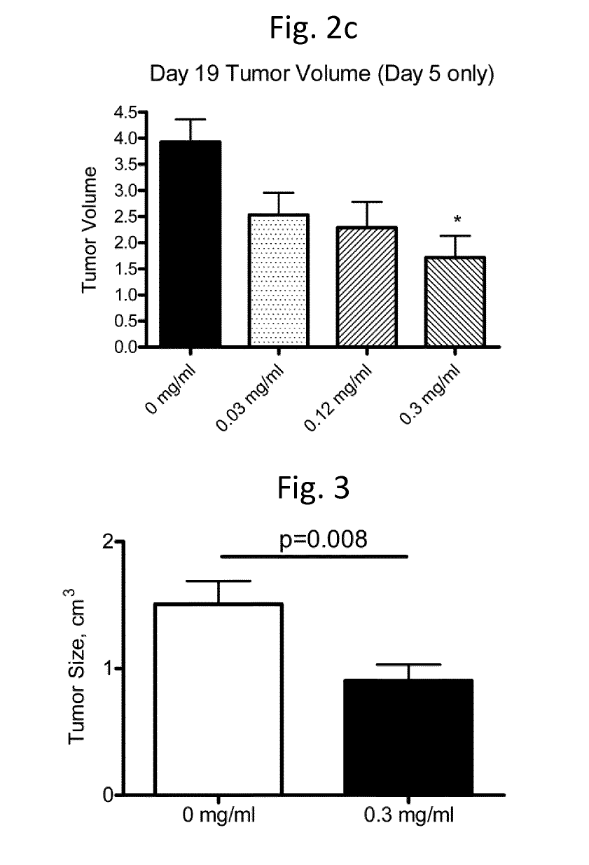

Tumors were initially detected at day 9. Starting on day 19, some tumors had grown so large that the mice had to be sacrificed. The experiment was terminated on day 25. FIG. 1A shows that OPN administration at the time of tumor injection had no significant effect on the size of the tumors up to 17 days. When OPN administration was initiated five days after tumor cell injection, however, there was a statistically significant decrease in the size of the tumor in both the 0.12 mg/ml and 0.3 mg/ml groups, at 15 and 17 days, respectively (FIG. 1b). FIG. 2 shows a comparison of tumor sizes in all the groups on days 15, 17 and 19; significant differences are indicated. FIG. 3 shows the mean tumor size of control and OPN-fed mice, combined results from three independent experiments. N=30 (0 mg/ml OPN) or 32 (0.3 mg/ml OPN).

These results clearly demonstrate that bovine OPN preparation, administered orally, can suppress the rate of growth of cancer tumors and possibly even prevent tumor growth. The amount of protein administered to the mice, about 1.5 mg, does not significantly alter their total protein intake, nor is there any effect of this treatment on mouse weight. Therefore, we conclude that this treatment has a specific effect on tumor or associated host cells that slows the growth of the tumor. Dosing experiments suggest that the highest dose of OPN preparation used, 0.3 mg/ml is most effective, and even higher doses may have larger effects on tumor growth.

### Example 2

### Effect of Orally Administered OPN on Primary Tumor Growth and Metastasis in a Mouse Model of Breast Cancer

4T1 cells are transformed mouse mammary epithelial cells (Aslakson 1992) that form metastatic tumors after orthotopic injection into the mouse mammary gland (Lelekakis 1999). This spontaneous metastasis formation—primarily to the lungs although other tissues can be involved—is a key feature of these cells that more faithfully reflects the development of metastases in human cancers than direct injection metastasis models. Accordingly, these cells are widely used as a model for aggressive human breast cancer. 4T1 cells also express high levels of OPN (Mi 2004), which is required for maximal tumor growth.

A. Mouse tumor development. 4T1 cells will be obtained from ATCC. In initial experiments, expression of OPN will be confirmed and cells will be tested for mycoplasma and remediated if necessary. Cells will be harvested while in exponential growth, washed, and 1×10⁵ cells will be injected into the mammary fat pad of 36 syngeneic Balb/c mice. The mice will be randomized into three groups, two of which will receive 0.3 mg/ml OPN in drinking water starting five days after tumor cell injection. The 4T1 cells form tumors rapidly: tumors are expected to be detected after 7-10 days, with maximal tumor volume reached by 25-30 days. Control mice and one group of OPN-fed mice (n=12 per group) will be sacrificed when any one mammary tumor reaches 20% of body weight, or if any pathology is detected. If, as we expect, the growth of the primary tumors will be suppressed in the OPN-fed animals, the second group of OPN-fed mice will be maintained until their tumors reach 20% of body weight. At sacrifice, a necropsy will be performed and sites of metastasis will be noted. Blood, tumors, lungs, and other metastatic tissues will be collected.

B. Analysis of mouse tissues. Primary tumors will be divided into three crosswise sections for cryopreservation, formaldehyde fixation and biochemical analyses. Lungs will be fixed for determination of metastatic burden, which will be assessed by counting surface nodules, and by histological examination of several lung sections. The number of surface metastases per mouse will be compared between mice which have been subjected to oral administration of OPN and control mice as the primary outcome. If differences in primary tumor growth rate are seen, blood vessel density and vessel morphology in different tumors will be assessed by CD31 and vWF staining, and lymphatic vessel density by staining for LYVE-1. If time allows, aspects of VEGF signalling will be analyzed: expression of various VEGF isoforms, expression of VEGF receptors, and the level of phosphorylation of those receptors will be assessed by western blot to test the hypothesis that oral administration of OPN causes altered VEGF signalling. These analyses will be carried out in both primary tumors and in metastatic lesions.

C. Expected results. Our hypotheses and previous data suggest that oral administration of OPN will suppress the growth of the primary mammary tumors in mice injected with 4T1 cells. If this is the case, and the corresponding metastatic burden is lower in OPN-fed mice, an additional group of OPN-fed mice will be included, in which tumors will be allowed to grow to the same size as control tumors, when they will be sacrificed and the metastatic burden determined. If lower numbers of metastases are seen in these animals as compared to controls, we will conclude that oral administration of OPN has a direct effect on metastasis. We anticipate that oral administration of OPN may result in increased blood vessel size, accompanied by increased activity of the VEGF signalling pathway.

### Example 3

### Functional Analysis of Peptides Derived from Orally Administered OPN

The inventors have reasons to believe that it is not OPN as such which provides the tumor suppressing effect, but rather relatively small peptide fragments which are formed by protease digestion of OPN.

In a previous experiment the inventors have detected osteopontin peptides in the plasma of mice fed with OPN using a competition ELISA for bovine OPN: the assay was designed to detect any peptides that can block interaction of intact bovine OPN with the polyclonal antibody used. Plasma from mice fed 50 mg of OPN over 10-30 minutes was analyzed using this ELISA. Peak antibody reactivity was detected at 1-4 hours after feeding, with the highest mean levels being close to 5000 ng/ml.

Example 3 identifies and tests the biologic activity of the bioactive peptides that mediate the tumor suppressive effect of oral administration of OPN. The hypothesis to be tested is that peptides derived from orally administered OPN are produced by the action of digestive enzymes in the stomach and small intestine, and that bioactive peptides are absorbed and accumulate in the circulation. To test this hypothesis, OPN will be fragmented using an in vitro model of digestion, and the resultant peptide preparations tested for the ability to suppress tumor growth by direct injection into mice, bypassing the enteral digestion system.

Several studies have described models of in vitro digestion of food-based proteins, mostly in order to understand their role in foodbased allergic responses (Wickham 2009). These models incorporate an initial pepsin digestion in acidic conditions to simulate gastric processes, followed by neutralization and reaction with pancreatic enzymes including trypsin and chymotrypsin simulating protein degradation in the small intestine. Additional pancreatic components such as lipases and bile salts are also frequently included, but these seem less relevant to the digestion of the isolated osteopontin protein, and will be omitted in initial experiments.

### A. Preparation of OPN Digests.

Sequential digestion with the digestive enzymes will be performed as previously described (Martos 2010.; Matsui 2002.). 500 mg of bovine osteopontin (or cGMP) will be dissolved in 5 ml sterile water, and the pH adjusted to 3 with 20% HCl, because the pH of the mouse gastric contents are apparently maintained at about pH 3-4 (Baumgartner 2002). This material will be digested with pepsin at 0.4 mg/ml for 4 hours at 37° C. The pH will then be readjusted to 6.8 using 20% NaOH and chymotrypsin and trypsin will each be added at 0.2 mg/ml and digestion will continue for 4 hours at 37° C. The digested material will be heated to 97° C. for 15 minutes, then centrifuged and the supernatant filtered through a 0.2 μm filter and lyophilized in 25 mg aliquots. Each aliquot will be dissolved in 1.4 ml sterile water and 0.1 ml injected intraperitoneally to groups of tumor-bearing mice. cGMP is a fragment of bovine κ-casein produced by Arla Foods Ingredients that will be used as a non-specific control for IP injection of peptides. This is an appropriate control because it is available in large quantities, and like OPN it is isolated from bovine milk, and it is hydrophilic, phosphorylated and glycosylated.

### B. Determination if OPN Digest is Effective in Suppressing Tumor Growth

This part of the experiment will determine if the injection of the peptides derived from the in vitro digestion directly into mice can suppress tumor growth. Intra-peritoneal (IP) injection is a common method used for introducing peptides: it is an effective route for administering peptide pharmaceuticals, and is low stress for the mice. The maximal possible dose, if all the administered OPN were ingested and absorbed at one time, would be 1-2 mg (based on the weight of the intact protein). The midpoint of this range, 1.5 mg, will be chosen as a starting dose. Since this is a relatively large amount of protein for IP injection, a pilot experiment will be conducted to confirm the safety of the protein digest. Groups of 5 mice (without tumors) will be injected daily for 20 days with A) 1.5 mg of intact OPN, B) 1.5 mg of digested OPN, C) 1.5 mg of digested cGMP, D) enzyme mixture only, without protein substrate or E) vehicle only. Endotoxin levels in all preparations will be tested and adjusted to <1 EU/dose. Mice will be weighed every other day and examined for signs of distress. At sacrifice, plasma will be collected and tested for the presence of immunoreactive bovine OPN using the competition ELISA.

If, as expected, the pilot experiment indicates that the digested OPN can be safely injected into mice at this dose, then the effect of these preparations on tumor development will be tested. Female 129B6F1 mice (total of 60 mice) will be injected with 275-3-2 tumor cells (see Example 1). The mice will be randomized to six groups that will receive daily injections starting 5 days after tumor inoculation as follows A) 1.8 mg diBested OPN; B) 0.36 mg digested OPN; C)1.8 mg digested cGMP; D) vehicle only; including enzymes; E) OPN in the drinking water; and F) no treatment, water only. Tumor size will be measured every other day, and all mice will be sacrificed when the first tumor of any group reaches 20% of the body weight of the animal, which is expected to be by 17-19 days. Plasma and tumor samples will be collected from the mice at sacrifice for further analysis.

Concurrently, digested proteins will be analyzed by SDS PAGE, size exclusion chromatography, C18 reverse phase-HPLC and mass spectrometry to identify some or all of the actual peptides produced by the digestion process. We will be especially interested in peptides that absorb at 280 (containing aromatic amino acids) since these may contain parts of the SVAYGLK sequence. If the digests are effective in suppressing tumor growth, then we will have identified a number of candidate peptides; conversely, if the digest has no effect then we can rule out the identified peptides as potential candidates.

### C. Identification of Active Peptides in OPN Digests

If the digested material is able to significantly inhibit tumor growth, then the digested peptides will be fractionated to begin to identify the active component(s). Peptides will be separated by size on a size-calibrated Superdex peptide column into three fractions. Based on the predicted cleavage pattern, we expect the peptide GDSVAY to be formed, with a molecular weight of 610 (or 700 if phosphorylated). Since this is a likely candidate, column fractions with size from 450-800 will be pooled, as well as fractions <450 and >800. The capacity of this column is about 2.5 mg/column, so several column runs will be performed and pooled to obtain sufficient material for mouse injections: this is practical only if a dose of 0.36 mg/mouse is effective. Alternatively, the peptide digest will be fractionated by preparative reverse-phase HPLC. The exact fractionation method to be employed will depend on the results of early analytic experiments and will be determined in consultation with experts at the FAS Center for Systems Biology Mass Spectrometry and Proteomics Resource Laboratory at Harvard University. A total of three pools of separated peptides will be prepared and used for injection into tumor bearing mice as described above. If positive results are obtained with one fraction, a final experiment testing further fractionated peptides will be performed.

### D. Expected Results

We anticipate that the peptide preparations will be well tolerated by the mice, and that the lower dose will be effective in suppressing tumor growth. By the end of the second round of purification, we expect that we will have narrowed down the candidate effective peptides to 3-10 individual species. Final identification of the effective peptides is performed by preparing purified or synthetic samples of each of the candidate peptides and repeating Steps B and C of this Example using the purified samples.

### Example 4

### Determination of the Level OPN in a Tumor

This example describes how the level of OPN in a tumor is determined.

### Sample Preparation:

A sample of the tumor in question is obtained and subsequently flash frozen and homogenized (about 50 mg tumor sample/0.25 ml buffer in Cell Lysis Buffer (Cell Signalling Technologies, Cat #9803) in the presence of protease inhibitors (Roche Applied Science cat #05 892 791 001). Homogenization is performed in 1.8 ml eppendorf tubes using plastic pestles on ice, for 30 secs to 1 minute.

### Determination of Total Protein:

The protein concentration of the tumor extract is determined using a bicinchoninic acid (BCA) assay kit (Pierce Biotechnology Cat #23227).

### Determination of the OPN Level:

The OPN level of the tissue extract is determined by ELISA using antibodies raised against the recombinant OPN which is native to the subject from which the blood sample is taken.

In the case of human subjects, monoclonal antibodies against human serum OPN should be used. One may for example use the Assay Designs Kit (Enzo Life Sciences, Cat# ADI-900-142) according to the manufacturer's instructions. The assay is calibrated with controlled samples of purified recombinant human OPN in varying concentrations from 2-32 ng/ml.

In the case of murine subjects, monoclonal antibodies against murine serum OPN should be used. One may for example use the Mouse Osteopontin ELISA Duoset Kit (R&D Systems, Cat # DY441) according to the manufacturer's instructions. The assay is calibrated with controlled samples of purified recombinant murine OPN in varying concentrations from 31-1000 pg/ml.

The resulting level of OPN in the tumor is presented as nanogram OPN per microgram total protein in the tumor sample.

### Example 5

### Determination of the Concentration of OPN in Plasma

This example describes how the concentration of OPN in plasma is determined.

### Sample Preparation:

Plasma is prepared from blood collected from the subject in the presence of 1.8 mg NaEDTA per mL blood.

### Determination of the OPN Concentration:

The concentration of OPN is determined by ELISA using monoclonal antibodies raised against recombinant OPN which is native to the subject from which the blood sample is taken.

In the case of human subjects, monoclonal antibodies against human serum OPN should be used. One may for example use the Assay Designs Kit (Enzo Life Sciences, Cat#ADI-900-142) according to the manufacturer's instructions. The level of OPN in normal human plasma ranges from 14-45 ng/ml. The assay is calibrated with controlled sampies of purified recombinant human OPN in varying concentrations from 2-32 ng/ml.

In the case of murine subjects, monoclonal antibodies against murine serum OPN should be used. One may for example use the Mouse Osteopontin ELISA Duoset Kit (R&D Systems, Cat # DY441) according to the manufacturer's instructions. The assay is calibrated with controlled samples of purified recombinant murine serum OPN in varying concentrations from 31-1000 pg/ml.

The resulting level of OPN in the plasma is presented as nanograms OPN per mL plasma.

### Example 6

### Anti-Tumor Effect of OPN-Derived Peptide Fragments

This trial was performed to document anti-tumor effects of OPN-derived peptide fragments. Three peptide fragments identified by in vitro proteolysis as described in Example 3 as potential products of intestinal digestion were synthesized: GDS*VAY (*=phosphorylated on serine), GDSVA, and VAYGL.

The peptide fragments were dissolved in dilute HCL, lyophilized to remove residual trifluoroacetic acid, and dissolved (all three peptides in one solution) at 3.6 mg/ml of each peptide fragment in saline. Mice were injected on the right flank with 275-3-2 tumor cells (5×10⁴ cells per mouse) on day zero, and were injected daily (intraperitoneal) with 0.1 ml of peptide fragment solution starting five days after tumor cell injection. Total peptide fragment dose was 54 mg/kg body weight per day (18 mg/kg of each peptide fragment). The control animals received no injections.

Both groups of mice were fed an irradiated standard laboratory rodent chow, comprising 20% protein and 4.5% fat.

The tumors of each mouse were measured every other day with calipers, noting the tumor-length, l, and the tumor width, w, and the tumor size was calculated as:

4/3·pi·(l/2·(w/2)²).

At sacrifice, tumors were excised from treated and control mice and weighed.

The statistical significance was calculated by t-test.

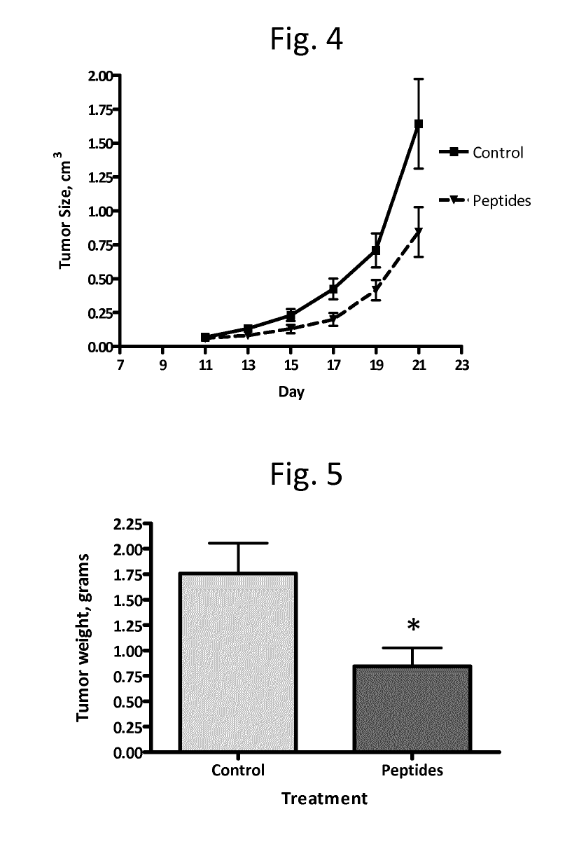

The results of the trial are shown in FIGS. 4 and 5. Here it is seen that the administration of peptide fragment solution clearly reduces the growth of the cancer tumor by approx. 50% at the selected dosage.

### Example 7

### Anti-Tumor Effect of OPN-Derived Peptide Fragments—Compared to Scrambled Peptides

This in vivo trial was performed to document anti-tumor effects of OPN-derived peptide fragments using scrambled peptides as controls. The peptides VAYGL, GDSVA, GDS*VAY and two scrambled control peptides (KYAGSDGVL and KYAGS*DGVL) (*indicates phosphorylated on serine) were synthesized and purified by HPLC. Each peptide was dissolved in 50 mM HCl, lyophilized and then subsequently dissolved in PBS (neutral pH) to obtain a total peptide concentration of 10.8 mg/ml.

The trial involved 50 mice (same type as in Example 1) divided into 5 groups (10 mice per group). At day 0 all the mice received subcutaneous injections of 275-3-2 tumor cells (5×10⁴ cells per mouse). Starting on day 5, the mice were given daily intraperitoneal peptide injections (0.1 ml aqueous peptide formulation/mouse/day). Tumor size was measured every other day with calipers (see Example 6). At sacrifice, the tumors were excised and weighed and collected for in vitro analysis.

| | Administered | Concentration of | Dosage (mg/kg |
|---|---|---|---|
| Group | peptides | peptides (mg/mL) | body weight) |
| 1 | VAYGL | 10.8 | approx. 54 |
| 2 | GDSVA | 10.8 | approx. 54 |
| 3 | GDS*VAY | 10.8 | approx. 54 |
| 4 | VAYGL, GDSVA and | 3.6 | approx. 54 |
| | GDS*VAY | (of each peptide) | |
| 5 | KYAGSDGVL and | 5.4 | approx. 54 |
| | KYAGS*DGVL | (of each peptide) | |

### Results

The tumor growth rate in the mice of groups 1-4 was significantly lower than that in the mice of group 5, which involved administration of the scrambled peptide controls. At sacrifice the tumor sizes of the tumors harvested from the mice, which had received OPN-derived peptides, were reduced by approx. 50% relative to the tumors of mice, which had received the scrambled peptides.

Thus, the present trial confirms the findings of the trial described in Example 6 and documents that individual active molecules of the present invention are effective as well.

### REFERENCES

Aslakson 1992 Aslakson, C. J., and Miller, F. R. (1992). Selective events in the metastatic process defined by analysis of the sequential dissemination of subpopulations of a mouse mammary tumor. Cancer Res 52, 1399-1405.
Anborgh 2009 Anborgh, P. H., Wilson, S. M., Tuck, A. B., Winquist, E., Schmidt, N., Hart, R., Kon, S., Maeda, M., Uede, T., Stitt, L. W., et al. (2009). New dual monoclonal ELISA for measuring plasma osteopontin as a biomarker associated with survival in prostate cancer: clinical validation and comparison of multiple ELISAs. Clin Chem 55, 895-903.
Baumgartner 2002 Baumgartner, H. K., Kirbiyik, U., Coskun, T., Chu, S., and Montrose, M. H. (2002). Endogenous cyclo-oxygenase activity regulates mouse gastric surface pH. J Physiol 544, 871-882.
Butler 1996 Butler W T, et al. 1996. Osteopontin. In Bilezekian J P, Rai L G, Rodan G A (eds.) Principles of bone biology. Academic Press, San Diego, Calif., U.S.A., pp. 167-181.
Denhardt 1995 Denhardt D T, et al. (eds.) 1995. Osteopontin: role in cell signalling and adhesion. Ann. N.Y. Acad. Sci., 760
Feng 2000 Feng, F., and Rittling, S. R. (2000). Mammary tumor development in MMTV-c-myc/MMTV-v-Ha-ras transgenic mice is unaffected by osteopontin deficiency. Breast Cancer Res. Treat. 63, 71-79.
Franzen 1985 Franzen A, Heineg 1985. Biochem. J. 232:715-724
Lelekakis 1999 Lelekakis, M., Moseley, J. M., Martin, T. J., Hards, D., Williams, E., Ho, P., Lowen, D., Javni, J., Miller, F. R., Slavin, J., et al. (1999). A novel orthotopic model of breast cancer metastasis to bone. OlinExpMetastasis 17, 163-170.
Martos 2010 Martos, G., Contreras, P., Molina, E., and Lopez-Fandino, R. (2010). Egg white ovalbumin digestion mimicking physiological conditions. 3 Agric Food Chem 58, 5640-5648.
Matsui 2002 Matsui, T., Tamaya, K., Seki, E., Osajima, K., Matsumoto, K., and Kawasaki, T. (2002). Val-Tyr as a natural antihypertensive dipeptide can be absorbed into the human circulatory blood system. Clin Exp Pharmacol Physiol 29, 204-208.
Mi 2004 Mi, Z., Guo, H., Wai, P. Y., Gao, C., Wei, J., and Kuo, P. C. (2004). Differential osteopontin expression in phenotypically distinct subclones of murine breast cancer cells mediates metastatic behavior. J Biol Chem 279, 46659-46667.
Nelson 2005 Nelson et al, Annals of Internal Medicine, Volume 143, Number 5, 6 Sep. 2005, p. 362-379
Rittling 1997 Rittling, S. R., and Novick, K. E. (1997). Osteopontin expression in mammary gland development and tumorigenesis. Cell Growth and Differentiation 8, 1061-1069.
Rittling 2002 Rittling, S. R., Chen, Y., Feng, F., and Wu, Y. (2002). Tumorderived osteopontin is soluble, not matrix associated. Journal of Biological Chemistry 277, 9175-9182.
Senger 1988 Senger D R, et al. 1988. Cancer Res. 48: 5770-5774 Sorensen 1994 Sorensen E S, et al. 1994. Protein Sci. 4:2040-2049
Tomayko 1989 Tomayko and Reynolds; Cancer Chemotherapy and Pharmacology (1989), Vol. 24, No. 3, p. 148-154)
Tuck 1998 Tuck, A. B., O'Malley, F. P., Singhal, H., Harris, J. F., Tonkin, K. S., Kerkvliet, N., Saad, Z., Doig, G. S., and Chambers, A. F. (1998). Osteopontin expression in a group of lymph node negative breast cancer patients. International Journal of Cancer 79, 502-508.
Wickham 2009 Wickham, M., Faulks, R., and Mills, C. (2009). In vitro digestion methods for assessing the effect of food structure on allergen breakdown. Mol Nutr Food Res 53, 952-958.
Wu 2000 Wu, Y., et al., Br. J. Cancer (2000) 83: 156-163

1-24. (canceled)

25. An active agent comprising one or more isolated, pharmaceutically active molecules, said one or more isolated, pharmaceutically active molecules comprising an amino acid sequence of at least three consecutive amino acid resides from SEQ ID NO. 22, said amino acid sequence containing at most 15 consecutive amino acids taken from position 147 to position 170 of SEQ ID NO. 2, for use in the treatment or prevention of cancer involving at least one cancer tumor.

26. The active agent for use in the treatment or prevention according to claim 25 wherein the one or more isolated, pharmaceutically active, molecules according to any of the preceding claims have a molecular weight of at most 5 kg/mol.

27. The active agent for use in the treatment or prevention according to claim 25, wherein the isolated, pharmaceutically active, molecule comprises 3-10 amino acids.

28. The active agent for use in the treatment or prevention according to claim 25, wherein the amino acid sequence of the active molecule is selected from the group consisting of SEQ ID NO 7, 20 and 21.

29. The active agent for use in the treatment or prevention according to claim 25, wherein the active molecule consists essentially of the amino acid sequence.

30. The active agent for use in the treatment or prevention according to claim 25, wherein the active molecule is in the form of a peptide having a sequence selected from the group consisting SEQ ID No. 7, 20 and 21.

31. The active agent for use in the treatment or prevention according to claim 25, wherein the active molecule comprises at least one glycosylated amino acid residue.

32. The active agent for use in the treatment or prevention according to claim 25, wherein the C-terminal amino acid residue of the amino acid sequence of the active molecule is unmodified.

33. The active agent for use in the treatment or prevention according to claim 25, wherein the N-terminal amino acid residue of the amino acid sequence of the active molecule is unmodified.

34. The active agent for use in the treatment or prevention according to claim 25, for reducing the risk of or preventing metastasis in a subject having a cancer involving at least one cancer tumor.

35. The active agent according to claim 25, wherein the active agent is administered in a daily dosage in the range of about 0.05 mg/kg of body weight to about 5 g/kg of body weight of the subject treated.

36. The active agent according to claim 25, wherein the treatment or prevention is by oral administration.

37. The active agent according to claim 25, wherein the treatment or prevention is by parenteral administration.

38. A pharmaceutical composition comprising:
an active agent comprising one or more isolated, pharmaceutically active, molecule(s), said isolated, pharmaceutically active, molecule(s) comprising an amino acid sequence of at least three consecutive amino acid residues from SEQ ID NO. 22, said amino acid sequence containing at most 15 consecutive amino acids taken from position 147 to position 170 of SEQ ID NO. 2,
one or more additional therapeutic agent(s) comprising an anti-cancer agent, and
a pharmaceutically acceptable carrier.

39. An isolated, pharmaceutically active, molecule comprising an amino acid sequence, wherein the amino acid sequence is selected from the group consisting of SEQ ID NO 7, 20 and 21.

40. The isolated, pharmaceutically active, molecule according to claim 39 having a molecular weight of at most 5 kg/mol, preferably at most 1.5 kg/mol.

41. The isolated, pharmaceutically active, molecule according to claim 39, said the active molecule is in the form of a peptide having a sequence selected from the group consisting of SEQ ID NO 7, 20 and 21.

42. The isolated, pharmaceutically active, molecule according to claim 39 comprising at least one glycosylated amino acid residue.

43. The isolated, pharmaceutically active, molecule according to claim 39, wherein the C-terminal amino acid residue of the amino acid sequence of the active molecule is unmodified.

44. The isolated, pharmaceutically active, molecule according to claim 39, wherein the N-terminal amino acid residue of the amino acid sequence of the active molecule is unmodified.

45. The isolated, pharmaceutically active, molecule according to claim 39, said active molecule consisting essentially of the amino acid sequence.

46. An active agent comprising one or more isolated, pharmaceutically active, molecules according to claim 39 for use as a medicament.

47. A pharmaceutical composition comprising:
an active agent comprising one or more isolated, pharmaceutically active, molecule(s) according to claim 39, and
a pharmaceutically acceptable carrier.

48. A nutritional supplement comprising:
a nutritionally effective amount of an active agent comprising one or more isolated, pharmaceutically active, molecules(s) according to claim 39, and
one or more components selected from the group consisting of a carbohydrate source, a lipid source, and a protein source.
